(19) United States
(12) Patent Application Publication
(10) Pub. No.: US 2023/0145868 A1
(43) Pub. Date: May 11, 2023

(54) UREA INHIBITORS OF MICRO-RNA

(71) Applicant: Board of Regents, The University of Texas System, Austin, TX (US)

(72) Inventors: George CALIN, Pearland, TX (US); Philip JONES, Houston, TX (US); Barbara CZAKO, Bellaire, TX (US); Simone ANFOSSI, Houston, TX (US)

(21) Appl. No.: 18/145,595

(22) Filed: Dec. 22, 2022

Related U.S. Application Data
(63) Continuation of application No. PCT/US2021/043171, filed on Jul. 26, 2021.
(60) Provisional application No. 63/058,283, filed on Jul. 29, 2020.

(51) Int. Cl. A61K 31/416; A61P 35/00

(52) U.S. Cl. CPC A61K 31/416; A61P 35/00

(57) ABSTRACT

The present disclosure relates to compounds and methods which may be useful as inhibitors of the expression of miRNA, for use in the treatment or prevention of cancer.

This application is a bypass continuation of International Application No. PCT/US 2021/043171, filed Jul. 26, 2021, which claims the benefit of priority of U.S. Provisional Application No. 63/058,283, filed Jul. 29, 2020, the disclosures of which are hereby incorporated by reference as if written herein in their entireties.

Disclosed herein are new urea compounds and compositions and their application as pharmaceuticals for the treatment of disease. Methods of inhibition of micro-RNA (“microRNA” or “miRNA”) activity in a human or animal subject are also provided for the treatment diseases such as cancer.

A class of small noncoding RNA strands, termed microRNAs or miRNAs, produced by splicing from precursor miRNA transcripts, bind to target messenger RNAs (mRNAs), and lead to inhibition of translation or degradation. Aberrant expression of certain miRNAs has been associated with particular cancers. It is suspected that these miRNAs might play a role in cancer, either via oncogene activation, or by inactivation of tumor suppressor genes. Although their role in cancers have made miRNAs attractive targets for cancer chemotherapy, to date efforts to provide compounds capable of interfering with the activity of miRNAs have been unsuccessful. For example, nucleoside analogues capable of interacting with miRNAs have not yet proven effective, either due to low activity against miRNAs or unacceptable toxicity. There remains a need for the identification of compounds that can directly inhibit oncogenic miRNAs, and thus provide useful cancer therapy.

The oncogenic miRNA miR-10b has been identified in the metastatic process of tumor cells. High miR-10b expression is associated with metastasis in several human cancers, including breast cancer, pancreatic cancer, glioblastoma, bladder cancer, and liver cancer. Overexpression of miR-10b is observed in metastatic breast cancer, especially in lymph node metastasis compared to paired primary tumors. Similarly, study of xenograft models for breast cancer reveal that miR-10b overexpression promote metastasis. The behavior of downstream targets, including HOXD10, NF1, KLF4, and PTEN, may be modulated by the direct interaction with miR-10b. Proliferation, migration, and invasion of cancer cells upon inhibition of miR-10b expression in breast cancer models is observed. The effect is pronounced by combining miR-10b inhibitors with low-dose doxorubicin. Secretion of miR-10b from metastatic breast cancer cells can induce invasiveness in nonmalignant mammary epithelial cells. From a meta-analysis study, it was determined that high expression of miR-10b in cancer patients correlates with poor patient outcome. These factors suggest that targeting miR-10b, either with a single agent or in combination, could prove therapeutically effective against multiple types of cancer.

Novel compounds and pharmaceutical compositions, certain of which have been found to inhibit miRNA have been discovered, together with methods of synthesizing and using the compounds including methods for the treatment of miRNA-mediated diseases in a patient by administering the compounds.

### BRIEF DESCRIPTION OF THE SEVERAL VIEW OF THE DRAWINGS

FIG. 1 shows expression of miR-10b in four cancer lines according to tissue type: (i) brain (ii) pancreas (iii) stomach as logy normalized nanostring data from the Cancer Cell Line Encyclopedia (CCLE).

FIG. 2 shows the results of the luciferase assay for (a) MCF7 and (b) MDA-MB-231. (i) negative control, (ii) 2, (iii) 6, (iv) 4, (v) 9, (vi) 7, (vii) 8, (viii) 5 (ix) 10. Vertical axis =fold change from negative control.

FIG. 3 shows the effect of compounds 2 and 5 on expression of (I) miR-10b and (II) pre-miR-10b, normalized to U6. (i) DMSO; (ii) 2; (iii) 5. (a) AGS cell line; (b) AGS cell line: a second independent experiment 72 h post treatment; (c) AsPC1 cell line.

FIG. 4 shows the effect of compound 4 on expression of (a) miR-10b and (b) pre-miR-10b, normalized to U48, in the (I) AGS and (II) AsPC1 cell lines.

FIG. 5 shows the effect of compound 4 on expression of (a) miR-16, (b) miR-28, and (c) miR-182, normalized to U6, in the (I) AGS and (II) AsPC1 cell lines.

FIG. 6 shows the effect of compound 4 on expression of (a) miR-10a, (b) miR-21, and (c) miR-155, normalized to U6, in the (I) AGS and (II) AsPC1 cell lines.

FIG. 7 shows the effect of compound 2 on proliferation ability. (i) DMSO; (ii) 10 μM 2; (iii) 5 μM 2; (iv) 2.5 μM 2. (a) AGS cell line (horizontal axis=days) (b) AGS cell line (horizontal axis=hours) (c) AsPC1 cell line (horizontal axis=days).

FIG. 8 shows the effect of compound 4 on proliferation ability in the (I) AGS and (II) AsPC1 cell lines. (i) DMSO; (ii) 10 μM 4.

FIG. 9 shows IC₅₀ experiments at 48 h using 4, for the (a) U251 (b) LN229 (c) AGS (d) AsPC1 cell lines. Horizontal axis=log [4], μM; vertical axis=cell viability.

FIG. 10 shows IC₅₀ established in sequential doses at 24 h after treatment with 4. (a) U251 (b) LN229 (c) AGS (d) AsPC1. Horizontal axis=log [4], μM; vertical axis=cell viability.

FIG. 11 shows cell viability experiments using (i) DMSO, (ii) 5 μM 4, and (iii) 10 μM 4, for the (a) U251 (b) LN229 (c) AGS (d) AsPC1 cell lines. Horizontal axis=time (h); vertical axis=cell viability.

FIG. 12 shows RT-qPCR experiments on miR-10B expression in (a) U251 brain cancer (b) AGS gastric cancer and (c) AsPC1 pancreatic cancer cell lines in the presence of (i) DMSO and (ii) 10 μM 4. Vertical axis=miR-10B expression (normalized to U48).

FIG. 13 shows results from from clonogenic assay in the U251 brain cancer line (a) colony images (b) plot of colony count (vertical axis). (i) control (DMSO) (iii) 1 μM 4, (iii) 2.5 μM 4, and (iv) 5 μM 4.

FIG. 14 shows morphology of U251 brain cancer cells in the presence of (I) DMSO and (II) compound 4 at (a) 24 h and (b) 48 h, at 20× magnification.

FIG. 15 shows Annexin V/PI assay of (a) U251 and (b) AGS cell lines using (i) DMSO, (ii) 5 μM 4, and (iii) 10 μM 4. Vertical axis=% apoptotic cells. Also shown is (c) cell cycle analysis via flow cytometry using (i) DMSO and (ii) 5 μM 4. Vertical axis=% phase.

FIG. 16 shows (a) Western blot analysis for the expression of (i) PTEN, (ii) PDGF, and (iii) VEGF (loading control (iv) β-actin) in the presence of 4, in the brain cancer cell lines I 32 LN229 and II=U251, and (b) Western blot analysis for the expression of (i) Dicer and (ii) Drosha (loading control (iii) GAPDH) in the presence of 4, in the cancer cell types I=AsPC1, II=AGS, III=U251, and IV=LN229. Also shown is (c) proteomics analysis of PTEN expression in U251 with (i) DMSO (ii) 10 mM 4.

FIG. 17 shows Western blots for (I) AGS and (II) AsPC1 cell lines. (a) DMSO; (b) compound 4. (i) Drosha (ii) Dicer (iii) PTEN (iv) HOXD10 (v) β-actin.

FIG. 18 shows (I) Western blots for the AGS cell line (a) DMSO; (b) compound 2. (i) PTEN (ii) HOXD10 (iii) Drosha (iv) Dicer (v) β-actin. Also shown is effect of compound 2 on migration for the AGS cell line. (a) DMSO; (b) compound 2.

FIG. 19 shows the effect of compound 4 on migration for (I) AGS and (II) AsPC1 cell lines. (a) DMSO; compound 4.

FIG. 20 shows studies directed at the interaction of 4 with miR-10b (a) ¹H NMR of 100 mM 4 and 10 mM pre-miR-10b sequence, showing the —CH₃ signal (*) (b) Target detect 2D ¹H-¹H TOCSY of 50 μM pre-miR-10b sequence upon titration with up to 100 μ,M 4. (c) Schematic of hairpin site for miR-10b, with bases perturbed the most by the presence of 4 indicated in bold. (d) A model of the pre-miR-10b 3D structure, determined with FARFAR2, with the regions perturbed the most by the presence of 4 indicated in bold.

FIG. 21 shows studies directed at the interaction of 4 with iPSC-derived organoids. (a) Schematic of the process used to generate the organoids. (b) images (4× magnification of cerebral organoid (i) co-cultured with LN229 brain cancer cell line and (ii) 2 weeks post co-culture. (c) RT-qPCR examination of miR-10b expression in co-cultures of (ii) LN229 and (iii) U251 (in both cases (i)=control). Vertical axis=fold change normalized to U48. (d) RT-qPCR examination of miR-10b expression in co-cultures of (ii) LN229 and (iii) U251 (in both cases (i)=control) upon treatment with 4. Vertical axis miR-10b expression normalized to U48.

FIG. 22 shows representative in situ hybridization studies of (a) control, (b) LN229, and (c) U251 cerebral organoid model in the presence of DMSO, 5 μM 4, or 10 μM 4. (i) miR-10b expression (ii) U6 as positive control (iii) scramble miRNA as negative control.

FIG. 23 shows the effect of (I) DMSO and (II) 10 μM 4 on the expression of proteins of the PI3K/AKT pathway in (a) U251 and (b) AGS cell lines. (i) Akt (ii) Akt1 (iii) 1_pS473 (iv) Akt2 (v) Akt2_pS474 (vi) Akt2_pS473 (vii) Akt2_pT308 (viii) mTOR (ix) mMTOR_pS448 (x) PI3K-p110-a (xi) PI3K-p110-b (xii) PI3K-p85.

FIG. 24 shows a pathway enrichment analysis using the Hallmark Gene Set from the MSigDB database (a) U251 (b) AGS (i) HALLMARK)_APOPTOSIS (ii) HALLMARK_APICAL_JUNCTION (iii) HALLMARK_HYPDXIA (iv) HALLMARK_TNFA_SIGNALING_VIA_NFKB (v) HALLMARK_G2M_CHECKPOINT (vi) HALLMARK_MTORC1_SIGNALING (vii) HALLMARK_PI3K_AKT_MTOR_SIGNALING (viii) HALLMARK_UV_RESPONSE_DN (ix) HALLMARK_NOTCH_SIGNALING (x) HALLMARK_APOPTOSIS (xi) HALLMARK_APICAL_JUNCTION (xii) HALLMARK_E2F_TARGETS (xiii) HALLMARK_PI3K_AKT_MTOR_SIGNALING.

FIG. 25 shows an enrichment analysis using Gene Ontology (GO) (a) U251 (b) AGS. Horizontal axis (GeneRatio) ratio of differentially expressed genes. p.adjust=p-values adjusted for the false discovery rate (FDR) using the Benjamin-Hochberg correction. Biological Process (BP): (Ia) gland development (IIa) T cell activation (IIIa) epithelial cell proliferation (IVa) regulation of apoptotic signaling pathway (Va) cellular response to chemical stress. (Ib) gland development (IIb) reproductive structure development (IIIb) reproductive system development (IVb) regulation of apopototic signaling pathway (Vb) intrinsic apopototic signaling pathway. Cellular Component (CC): (VIa) nuclear chromatin (VIIa) focal adhesion (VIIIa) cell-substrate junction (IXa) chromosomal region (Xa) chromosome, telomeric region. (VIb) nuclear chromatin (VIIb) membrane raft (VIIIb) membrane microdomain (IXb) membrane region (Xb) DNA repair complex. Molecular Function (MF): (XIa) protein serine/threonine kinase activity (XIIa) phosphatase binding (XIIIa) ubiquitin-like protein ligase binding (XIVa) protein phosphatase binding (XVa) protein tyrosine kinase activity. (XIb) protein serine/threonine kinase activity (XIIb) ubiquitin-like protein ligase binding (XIIIb) ubiquitin protein ligase binding (XIVb) phosphatase binding (XVb) protein phosphatase binding.

### DETAILED DESCRIPTION

Provided herein is a compound of structural Formula I:

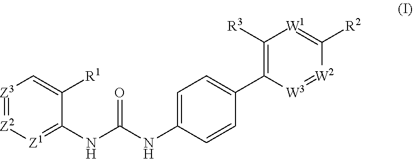

or a salt or tautomer thereof, wherein:
W¹ is chosen from CR⁴ and N;
W² is chosen from CR⁵ and N;
W³ is chosen from CR⁶ and N;
Z¹ is chosen from CR⁷ and N;
Z² is chosen from CR⁸ and N;
Z³ is chosen from CR⁹ and N;
R¹ and R² are independently chosen from H, CN, NH₂, OH, and halo;
R³ is chosen from H, CN, halo, hydroxy, alkyl, and alkoxy;
R⁴ is chosen from H, CN, halo, alkyl, and alkoxy;
or R⁴, if present, and R³, together with the intervening carbons, can form a 5-, 6-, or 7-membered cycloalkyl, heterocycloalkyl, aryl, or heteroaryl ring optionally substituted with one or more R¹⁰;
R⁵, R⁶, R⁷, R⁸, and R⁹ are independently chosen from H, CN, halo, alkyl, and alkoxy; and each R¹⁰ is independently chosen from CN, halo, OH, NH₂, and oxo.

The compounds disclosed herein possess useful miRNA-inhibiting activity, and may be used in the treatment or prophylaxis of a disease or condition in which miRNA plays an active role. Thus, also provided are pharmaceutical compositions comprising one or more compounds, or salts or tautomers thereof, disclosed herein together with a pharmaceutically acceptable carrier, as well as methods of making and using the compounds, or salts or tautomers thereof, and compositions. Also provided are methods for inhibiting miRNA. Also provided are methods for treating a miRNA-mediated disorder in a patient in need of such treatment, comprising administering to said patient a therapeutically effective amount of a compound, or salt or tautomer thereof, or composition according to the present disclosure. Also provided is the use of compounds, or salts or tautomers thereof, disclosed herein for use in the manufacture of a medicament for the treatment of a disease or condition ameliorated by the inhibition of microRNA.

In certain embodiments, R⁴ is chosen from H, CN, halo, C₁₋₆alkyl, and C₁₋₆alkoxy. In certain embodiments, R⁴ is chosen from H, CN, halo, and C₁₋₆alkyl. In certain embodiments, R⁴ is chosen from H, CN, and halo. In certain embodiments, R⁴ is chosen from H, F, Cl, and Br. In certain embodiments, R⁴ is chosen from H, F, and Cl. In certain embodiments, R⁴ is chosen from H and F. In certain embodiments, R⁴ is H.

In certain embodiments, R⁴ and R³, together with the intervening carbons, form a 5-, 6-, or 7-membered cycloalkyl, heterocycloalkyl, aryl, or heteroaryl ring optionally substituted with one or more R¹⁰. In certain embodiments, R⁴ and R³, together with the intervening carbons, form a 5- or 6-membered cycloalkyl, heterocycloalkyl, aryl, or heteroaryl ring optionally substituted with 1 or 2 R¹⁰. In certain embodiments, R⁴ and R³, together with the intervening carbons, form a 5-membered cycloalkyl, heterocycloalkyl, or heteroaryl ring optionally substituted with 1 or 2 R¹⁰. In certain embodiments, R⁴ and R³, together with the intervening carbons, form a 5-membered heterocycloalkyl or heteroaryl ring optionally substituted with 1 or 2 R¹⁰. In certain embodiments, R⁴ and R³, together with the intervening carbons, form a 5-membered heterocycloalkyl or heteroaryl ring optionally substituted with 1 R¹⁰.

In certain embodiments, each R¹⁰ is independently chosen from CN, OH, NH₂, and oxo. In certain embodiments, each R¹⁰ is independently chosen from OH, NH₂, and oxo. In certain embodiments, each R¹⁰ is independently chosen from OH and oxo. In certain embodiments, each R¹⁰ is NH₂.

In certain embodiments, at least one of Z¹, Z², and Z³ is N. In certain embodiments, at most two of Z¹, Z², and Z³ are N. In certain embodiments, at most one of Z¹, Z², and Z³ is N. In certain embodiments, exactly one of Z¹, Z², and Z³ is N.

In certain embodiments, Z¹ is CR⁷. In certain embodiments, Z¹ is N.

In certain embodiments, Z² is CR⁸. In certain embodiments, Z² is N.

In certain embodiments, Z³ is CR⁹. In certain embodiments, Z³ is N.

In certain embodiments, at least one of W¹, W², W³, Z¹, Z², and Z³ is N. In certain embodiments, at most two of W¹, W², W³, Z¹, Z², and Z³ are N. In certain embodiments, at most one of W¹, W², W³, Z¹, Z², and Z³ are N.

Also provided herein is a compound of structural Formula II:

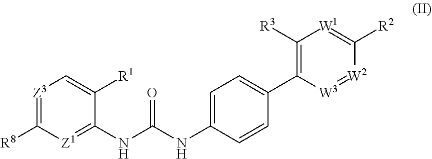

or a salt or tautomer thereof, wherein:
W¹ is chosen from CH and N;
W² is chosen from CR⁵ and N;
W³ is chosen from CR⁶ and N;
Z¹ is chosen from CR⁷ and N;
Z³ is chosen from CR⁹ and N;
R¹ and R² are independently chosen from H, CN, NH₂, OH, and halo;
R³ is chosen from H, CN, halo, hydroxy, alkyl, and alkoxy; and
R⁵, R⁶, R⁷, R⁸, and R⁹ are independently chosen from H, CN, halo, alkyl, and alkoxy.

In certain embodiments, R³ is chosen from H, CN, halo, hydroxy, C₁₋₆alkyl, and C₁₋₆alkoxy. In certain embodiments, R³ is chosen from H, CN, halo, and hydroxy. In certain embodiments, R³ is chosen from H, CN, F, Cl, and hydroxy. In certain embodiments, R³ is chosen from H, CN, F, and Cl. In certain embodiments, R³ is chosen from H, halo, and hydroxy. In certain embodiments, R³ is chosen from H and halo. In certain embodiments, R³ is chosen from H and F. In certain embodiments, R³ is H. In certain embodiments, R³ is F.
In certain embodiments, W¹ is CH. In certain embodiments, W¹ is N.

In certain embodiments, W² is CR⁵. In certain embodiments, W² is N.

In certain embodiments, W³ is CR⁶. In certain embodiments, W³ is N.

In certain embodiments, at least one of W¹, W², and W³ is N. In certain embodiments, at most one of W¹, W², and W³ is N. In certain embodiments, exactly one of W¹, W², and W³ is N.

In certain embodiments, exactly two of W¹, W², and W³ are N.

Also provided herein is a compound of structural Formula III:

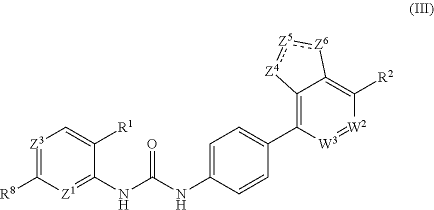

or a salt or tautomer thereof, wherein:
W² is chosen from CR⁵ and N;
W³ is chosen from CR⁶ and N;
Z¹ is chosen from CR⁷ and N;
Z³ is chosen from CR⁹ and N;
Z⁴ is chosen from CH(R^{10a}), C(R^{10a}), N(_{R}^{10a}), and N;
Z⁵ is chosen from CH(R^{10b}), C(R^{10ab}), N(R^{10b}, and N;
Z⁶ is chosen from CH(R^{10c}), C(R^{10c}), N(R^{10c}), and N;
R¹ and R² are independently chosen from H, CN, NH₂, OH, and halo;
R⁵, R⁶, R⁷, R⁸, and R⁹ are independently chosen from H, CN, halo, alkyl, and alkoxy; and
R^{10a}, R^{10b}, and R^{10c} are independently chosen from H, CN, halo, OH, NH₂, and oxo.

In certain embodiments, Z⁵ is C(R^{10b}). In certain embodiments, Z⁵ is N.

In certain embodiments, R^{110b}) is chosen from H, halo, OH, NH₂, and oxo. In certain embodiments, R^{10b} is chosen from H, OH, NH₂, and oxo. In certain embodiments, R^{10b} is chosen from H, OH, and oxo. In certain embodiments, R^{10b} is chosen from OH and oxo. In certain embodiments, R^{10b} is H.

In certain embodiments,
Z⁴ is N(R^{10a}); and
Z⁶ is C(R^{10c}).

In certain embodiments,
Z⁴ is C(R^{10a}); and
Z⁶ is N(R^{10c}).

In certain embodiments, Z⁴ is chosen from N(R^{10a}) and N.

In certain embodiments, Z⁶ is chosen from N(R^{10a}) and N.

Also provided herein is a compound of structural Formula IV:

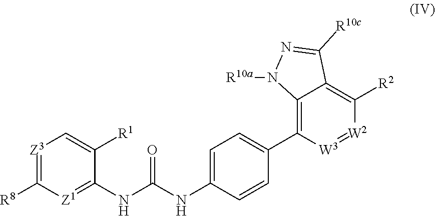

or a salt or tautomer thereof, wherein:
W² is chosen from CR⁵ and N;
W³ is chosen from CR⁶ and N;
Z¹ is chosen from CR⁷ and N;
Z³ is chosen from CR⁹ and N;
R¹ and R² are independently chosen from H, CN, NH₂, OH, and halo;
R⁵, R⁶, R⁷, R⁸, and R⁹ are independently chosen from H, CN, halo, alkyl, and alkoxy; and
R^{10a} and R^{10c} are independently chosen from H, CN, halo, OH, NH₂, and oxo.

Also provided herein is a compound of structural Formula V:

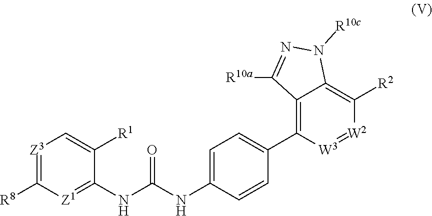

or a salt or tautomer thereof, wherein:
W² is chosen from CR⁵ and N;
W³ is chosen from CR⁶ and N;
Z¹ is chosen from CR⁷ and N;
Z³ is chosen from CR⁹ and N;
R¹ and R² are independently chosen from H, CN, NH₂, OH, and halo;
R⁵, R⁶, R⁷, R⁸, and R⁹ are independently chosen from H, CN, halo, alkyl, and alkoxy; and
R^{10a} and R^{10b} are independently chosen from H, CN, halo, OH, NH₂, and oxo.

Also provided herein is a compound of structural Formula VI:

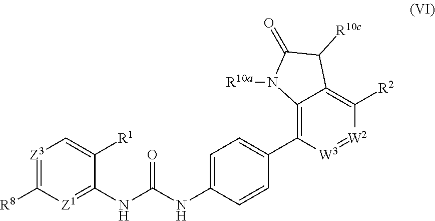

or a salt or tautomer thereof, wherein:
W² is chosen from CR⁵ and N;
W³ is chosen from CR⁶ and N;
Z¹ is chosen from CR⁷ and N;
Z³ is chosen from CR⁹ and N;
R¹ and R² are independently chosen from H, CN, NH₂, OH, and halo;
R⁵, R⁶, R⁷, R⁸, and R⁹ are independently chosen from H, CN, halo, alkyl, and alkoxy; and

R^{10a} and R^{10c} are independently chosen from H, CN, halo, OH, NH₂, and oxo.

In certain embodiments, R^{10a} and R^{10c} are independently chosen from H, CN, halo, OH, and NH2. In certain embodiments, R^{10a} and R^{10c} are independently chosen from H, halo, OH, and NH₂. In certain embodiments, R^{10a} and R^{10c} are independently chosen from H, OH, and NH₂. In certain embodiments, R^{10a} and R^{10c} are independently chosen from H and NH₂.

In certain embodiments, at least one of R^{10a} and R^{10c} is not H. In certain embodiments, exactly one of R^{10a} and R^{10c} is not H. In certain embodiments, exactly one of R^{10a} and R^{10c} is NH₂.

In certain embodiments, R^{10a} is H. In certain embodiments, R^{10a} is NH₂.

In certain embodiments, R^{10c} is H. In certain embodiments, R^{10c} is NH₂.

In certain embodiments,
R^{10a} is chosen from H, halo, OH, and NH₂; and
R^{10b} is H.

In certain embodiments,
R^{10a} is H; and
R^{10c} is chosen from H, halo, OH, and NH₂.

In certain embodiments, W² is CR⁵. In certain embodiments, W² is N.

In certain embodiments, W³ is CR⁶. In certain embodiments, W³ is N.

In certain embodiments, at least one of W² and W³ is N. In certain embodiments, exactly one of W² and W³ is N. In certain embodiments, at most one of W² and W³ is N.

In certain embodiments, Z¹ is CR⁷. In certain embodiments, Z¹ is N.

In certain embodiments, Z³ is CR⁹. In certain embodiments, Z³ is N.

In certain embodiments, at least one of Z¹ and Z³ is N. In certain embodiments, at most one of Z¹ and Z³ is N. In certain embodiments, exactly one of Z¹ and Z³ is N.

In certain embodiments, R¹ is chosen from H, CN, NH₂, and halo. In certain embodiments, R¹ is chosen from H, CN, and halo. In certain embodiments, R¹ is chosen from H, F, Cl, and Br. In certain embodiments, R¹ is chosen from H, F, and Cl. In certain embodiments, R¹ is chosen from H and F. In certain embodiments, R¹ is H. In certain embodiments, R¹ is F.

In certain embodiments, R² is chosen from H, CN, NH₂, and halo. In certain embodiments, R² is chosen from H, NH₂, and halo. In certain embodiments, R² is chosen from H, NH₂, F, Cl, and Br. In certain embodiments, R² is chosen from H, NH₂, F, and Cl. In certain embodiments, R² is chosen from H, NH₂, and F. In certain embodiments, R² is chosen from H and F. In certain embodiments, R² is NH₂. In certain embodiments, R² is H. In certain embodiments, R² is F.

In certain embodiments, R⁵ and R⁶ are chosen from H, CN, halo, C₁₋₆alkyl, and C₁₋₆alkoxy. In certain embodiments, R⁵ and R⁶ are independently chosen from H, F, and Cl. In certain embodiments, R⁵ and R⁶ are independently chosen from H and F. In certain embodiments, R⁵ and R⁶ are H

In certain embodiments, R⁵ is chosen from H, CN, halo, and C₁₋₆alkyl. In certain embodiments, R⁵ is chosen from H, CN, and halo. In certain embodiments, R⁵ is chosen from H, F, Cl, and Br. In certain embodiments, R⁵ is chosen from H, F, and Cl. In certain embodiments, R⁵ is chosen from H and F. In certain embodiments, R⁵ is H.

In certain embodiments, R⁶ is chosen from H, CN, halo, and C₁₋₆alkyl. In certain embodiments, R⁶ is chosen from H, CN, and halo. In certain embodiments, R⁶ is chosen from H, F, Cl, and Br. In certain embodiments, R⁶ is chosen from H, F, and Cl. In certain embodiments, R⁶ is chosen from H and F. In certain embodiments, R⁶ is H.

In certain embodiments, R⁷ and R⁹ are independently chosen from H, CN, halo, C₁₋₆alkyl, and C₁₋₆alkoxy. In certain embodiments, R⁷ and R⁹ are independently chosen from H, CN, halo, CH₃, and OCH₃. In certain embodiments, R⁷ and R⁹ are independently chosen from H, CN, halo, and CH₃. In certain embodiments, R⁷ and R⁹ are independently chosen from H, F, Cl, Br, and CH₃. In certain embodiments, R⁷ and R⁹ are independently chosen from H, F, Cl, and CH₃. In certain embodiments, R⁷ and R⁹ are independently chosen from H, F, and CH₃. In certain embodiments, R⁷ and R⁹ are independently chosen from H and CH₃. In certain embodiments, R⁷ and R⁹ are independently chosen from H, CN, and halo. In certain embodiments, R⁷ and R⁹ are independently chosen from H, F, and Cl. In certain embodiments, R⁷ and R⁹ are independently chosen from H and F.

In certain embodiments, at least one of R⁷ and R⁹ is not H. In certain embodiments, at most one of R⁷ and R⁹ is not H. In certain embodiments, exactly one of R⁷ and R⁹ is not H. In certain embodiments, wherein R⁷ and R⁹ are H.

In certain embodiments, R⁷ is chosen from H, CN, halo, CH3, and OCH₃. In certain embodiments, R⁷ is chosen from H, CN, halo, and CH₃. In certain embodiments, R⁷ is chosen from H, F, Cl, and CH₃. In certain embodiments, R⁷ is chosen from H and CH₃. In certain embodiments, R⁷ is chosen from H, F, and Cl. In certain embodiments, R⁷ is chosen from H and F. In certain embodiments, R⁷ is H. In certain embodiments, R⁷ is F.

In certain embodiments, R⁹ is chosen from H, CN, halo, CH₃, and OCH₃. In certain embodiments, R⁹ is chosen from H, CN, halo, and CH₃. In certain embodiments, R⁹ is chosen from H, F, Cl, and CH₃. In certain embodiments, R⁹ is chosen from H and CH₃. In certain embodiments, R⁹ is chosen from H, F, and Cl. In certain embodiments, R⁹ is chosen from H and F. In certain embodiments, R⁹ is H. In certain embodiments, R⁹ is F.

In certain embodiments, R⁸ is chosen from H, CN, halo, C₁₋₆alkyl, and C₁₋₆alkoxy. In certain embodiments, R⁸ is chosen from H, CN, halo, CH₃, and OCH₃. In certain embodiments, R⁸ is chosen from H, CN, halo, and CH₃. In certain embodiments, R⁸ is chosen from H, F, Cl, Br, and CH₃. In certain embodiments, R⁸ is chosen from H, F, Cl, and CH₃. In certain embodiments, R⁸ is chosen from H, F, and CH₃. In certain embodiments, R⁸ is chosen from H, CH₃, and OCH₃. In certain embodiments, R⁸ is chosen from CH₃ and OCH₃. In certain embodiments, R⁸ is chosen from H and CH₃. In certain embodiments, R⁸ is H. In certain embodiments, R⁸ is CH₃.

Also provided are embodiments, wherein any embodiment above may be combined with any one or more of these embodiments, provided the combination is not mutually exclusive.

As used herein, two embodiments are “mutually exclusive” when one is defined to be something which is different than the other. For example, an embodiment, wherein two groups combine to form a cycloalkyl is mutually exclusive with an embodiment in which one group is ethyl the other group is hydrogen. Similarly, an embodiment, wherein one group is CH₂ is mutually exclusive with an embodiment, wherein the same group is NH.

Also provided is a compound chosen from:

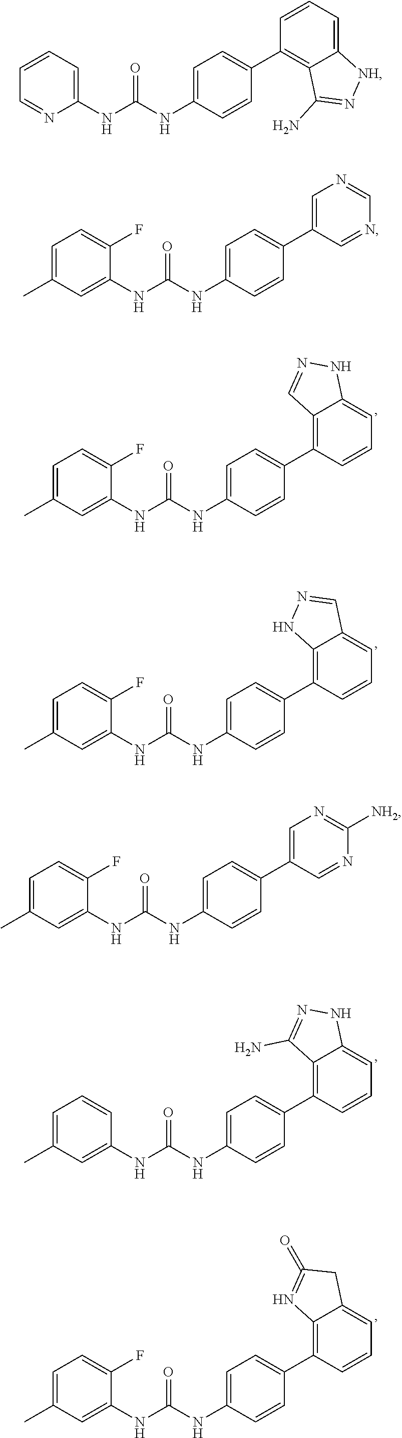

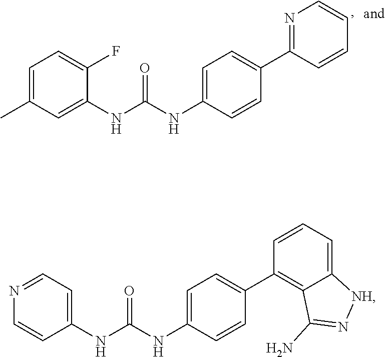 [SMILES: Cc1ccc(F)c(NC(=O)Nc2ccc(-c3ccccn3)cc2)c1 ; Nc1n[nH]c2cccc(-c3ccc(NC(=O)Nc4ccncc4)cc3)c12]

or a salt or tautomer thereof.

Also provided is a compound chosen from the Examples, or a salt or tautomer thereof, disclosed herein.

The present disclosure also relates to a method of inhibiting at least one miRNA function comprising the step of contacting miRNA with a compound as described herein, or a salt or tautomer thereof. The cell phenotype, cell proliferation, cell death or cell migration and metastatic activity of miRNA, change in biochemical output produced by active miRNA, expression of miRNA, or binding of miRNA with a natural binding partner may be monitored. Such methods may be modes of treatment of disease, biological assays, cellular assays, biochemical assays, or the like.

Also provided herein is a method of treatment of a miRNA-mediated disease comprising the administration of a therapeutically effective amount of a compound as disclosed herein, or a salt or tautomer thereof, to a patient in need thereof.

In certain embodiments, the disease is cancer. In some embodiments, the disease is an infectious disease, an immune disease, or a neurologic disease, or more generally any disease with abnormal levels of expression of miR-10b.

In certain embodiments, the method of treatment of a miRNA-mediated disease further comprises a non-chemotherapeutic method of treatment. In certain embodiments, the non-chemotherapeutic method of treatment is radiation therapy. In certain embodiments, administration of a compound as disclosed, or a salt or tautomer thereof, herein sensitizes the patient in need thereof to radiation therapy.

Also provided herein is a method of enhancing immunological activity comprising the administration of a therapeutically effective amount of a compound as disclosed herein, or a salt or tautomer thereof, to a patient in need thereof.

Also provided herein is a compound as disclosed herein, or a salt or tautomer thereof, for use as a medicament.

Also provided herein is a compound as disclosed herein, or a salt or tautomer thereof, for use as a medicament for the treatment of a miRNA-mediated disease.

Also provided is the use of a compound as disclosed herein, or a salt or tautomer thereof, as a medicament.

Also provided is the use of a compound as disclosed herein, or a salt or tautomer thereof, as a medicament for the treatment of a miRNA-mediated disease.

Also provided is a compound as disclosed herein, or a salt or tautomer thereof, for use in the manufacture of a medicament for the treatment of a miRNA-mediated disease.

Also provided is the use of a compound as disclosed herein, or a salt or tautomer thereof, for the treatment of a miRNA-mediated disease.

Also provided herein is a method of inhibition of miRNA comprising contacting miRNA with a compound as disclosed herein, or a salt or tautomer thereof.

Also provided herein is a method for achieving an effect in a patient comprising the administration of a therapeutically effective amount of a compound as disclosed herein, or a salt or tautomer thereof, to a patient, wherein the effect is chosen from cognition enhancement.

In certain embodiments, the miRNA-mediated disease is cancer, an infectious disease, an immune disease, or a neurologic disease, or more generally any disease with abnormal levels of expression of miR-10b.

Also provided is a method of modulation of a miRNA-mediated function in a subject comprising the administration of a therapeutically effective amount of a compound as disclosed herein, or a salt or tautomer thereof.

Another aspect of the present disclosure includes a method of reducing or eliminating the biological activity of microRNA in a cell or organism. In some embodiments, the method includes contacting a cell with a therapeutically effective amount of a compound as disclosed herein, or a salt or tautomer thereof, or any one or combination of pharmaceutical compositions described herein.

Also provided is a pharmaceutical composition comprising a compound as disclosed herein, or a salt or tautomer thereof, together with a pharmaceutically acceptable carrier.

In certain embodiments, the pharmaceutical composition is formulated for oral or parenteral administration. In certain embodiments, the pharmaceutical composition is formulated for oral administration. In certain embodiments, the pharmaceutical composition is formulated for parenteral administration.

In certain embodiments, the oral pharmaceutical composition is chosen from a tablet and a capsule.

### Abbreviations and Definitions

As used herein, the terms below have the meanings indicated.

When ranges of values are disclosed, and the notation “from n₁ . . . to n₂” or “between n₁ . . . and n₂” is used, where ni and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon group having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl groups include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether group, wherein the term alkyl is as defined below. Examples of suitable alkyl ether groups include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl group containing from 1 to 20 carbon atoms. In certain embodiments, said alkyl will comprise from 1 to 10 carbon atoms. In further embodiments, said alkyl will comprise from 1 to 8 carbon atoms. Alkyl groups may be optionally substituted as defined herein. Examples of alkyl groups include methyl, ethyl, n-propyl, isopropyl, n-butyl, isobutyl, sec-butyl, tert-butyl, pentyl, iso-amyl, hexyl, octyl, nonyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The terms “amido” and “carbamoyl,”as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings, wherein such polycyclic ring systems are fused together. The term “aryl” embraces aromatic groups such as phenyl, naphthyl, anthracenyl, and phenanthryl.

The terms “benzo” and “benz,” as used herein, alone or in combination, refer to the divalent group C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(0)NR′, group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group, wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronaphthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1.1.1]pentane, camphor, adamantane, and bicyclo[3.2.1]octane

The term “bicycloalkyl”, as used herein, alone or in combination, refers to a cyclic alkyl system that is characterized by the presence of two atoms, termed “bridgehead atoms” that are connected to each other via three bond pathways. “Bicycloalkyl” thus encompasses, by way of example, bicyclo[2.2.1]heptane, also known as norbornane, bicyclo[2.2.2]octane, bicyclo[2.2.0]hexane and bicyclo[3.3.0]octane.

The term “cycloalkoxy”, as used herein, alone or in combination, refers to a saturated, or partially unsaturated monocyclic, bicyclic, or tricyclic heterocyclic group containing at least one oxygen as a ring member. In some embodiments, said cycloalkoxy comprises 1, 2, or 3 heteroatoms as ring members. In some embodiments, said cycloalkoxy contains 1 or 2 heteroatoms as ring members. In some embodiments, said cycloalkoxy contains 1 oxygen as a ring member. In some embodiments, the heteroatoms in said heterocycloalkyl are independently chosen from nitrogen, oxygen, and sulfur. In some embodiments, the heteroatoms in said heterocycloalkyl are independently chosen from nitrogen and oxygen. In some embodiments, the heteroatoms in heterocycloalkyl are oxygen. In some embodiments, the heterocycloalkyl contains at least one aryl or heteroaryl ring. In some embodiments, the heterocycloalkyl does not contain either an aryl ring or a heteroaryl ring. Examples of cycloalkoxy groups include ethylene oxide, oxetane, tetrahydrofuran, 2,3-dihydrobenzofuran, dioxane, and morpholine.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “fluoroalkyl,” as used herein, alone or in combination, refers to an alkyl group having the meaning as defined above, wherein one or more hydrogens are replaced with a fluorine. Specifically embraced are monofluoroalkyl, difluoroalkyl and polyfluoroalkyl groups. Examples of fluoroalkyl groups include fluoromethyl, difluoromethyl, trifluoromethyl, 2,2,2-trifluoroethyl, pentafluoroethyl, heptafluoropropyl, difluoroethyl, difluoropropyl.

The term “halo,” or “halogen,” as used herein, alone or in combination, refers to fluorine, chlorine, bromine, or iodine.

The term “haloalkyl,” as used herein, alone or in combination, refers to an alkyl group having the meaning as defined above, wherein one or more hydrogens are replaced with a halogen. Specifically embraced are monohaloalkyl, dihaloalkyl and polyhaloalkyl groups. A monohaloalkyl group, for one example, may have an iodo, bromo, chloro or fluoro atom within the group. Dihalo and polyhaloalkyl groups may have two or more of the same halo atoms or a combination of different halo groups. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups, wherein heterocyclic rings are fused with aryl rings, wherein heteroaryl rings are fused with other heteroaryl rings, wherein heteroaryl rings are fused with heterocycloalkyl rings, or, wherein heteroaryl rings are fused with cycloalkyl rings. Examples of heteroaryl groups include pyrrolyl, imidazolyl, pyrazolyl, pyridyl, pyrimidinyl, pyrazinyl, pyridazinyl, triazolyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzoxazolyl, benzoxadiazolyl, benzothiazolyl, benzothiadiazolyl, benzofuryl, benzothienyl, chromonyl, coumarinyl, benzopyranyl, tetrazolopyridazinyl, thienopyridinyl, furopyridinyl, pyrrolopyridinyl and the like. Exemplary tricyclic heterocyclic groups include carbazolyl, phenanthrolinyl, dibenzofuranyl, acridinyl, phenanthridinyl, and the like.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently chosen from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited. The term “heterocycloalkyl”, as used herein, alone or in combination, is understood to encompass “heterobicycloalkyl”, as defined below.

The term “hydroxy,” as used herein, alone or in combination, refers to —OH.

The terms “oxy” or “oxa,” as used herein, alone or in combination, refer to —O—.

The term “oxo,” as used herein, alone or in combination, refers to ═O.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, (O)CH₃; CO₂CH₃, CO₂H, pyridinyl, thiophene, furanyl, lower carbamate, and lower urea. Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms,as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art.

Additionally, the compounds disclosed herein may exist as geometric isomers. The present disclosure includes all cis, trans, syn, anti, entgegen (E), and zusammen (Z) isomers as well as the appropriate mixtures thereof.

Additionally, compounds may exist as tautomers; all tautomeric isomers are provided by this disclosure. Tautomers include, but are not limited to: keto/enol tautomers, lactam/lactim tautomers, and amide/imidic acid tautomers. Also included are amidine tautomers having the following tautomeric equilibrium: R—C(═NHR¹)—NHR²/R—C(—NHR¹)═NHR².

Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “miRNA inhibitor” is used herein to refer to a compound that exhibits an IC₅₀ with respect to miRNA activity of no more than about 100 μM and more typically not more than about 50 μM, as measured in the miRNA assay described generally herein. “IC₅₀” is that concentration of inhibitor which reduces the activity of an enzyme, (e.g., miRNA) to half-maximal level. Certain compounds disclosed herein have been discovered to exhibit inhibition activity against miRNA. In certain embodiments, compounds will exhibit an IC₅₀ with respect to miRNA of no more than about 20 μM; in further embodiments, compounds will exhibit an IC₅₀ with respect to miRNA of no more than about 5 μM; in yet further embodiments, compounds will exhibit an IC₅₀ with respect to miRNA of not more than about 1 μM; in yet further embodiments, compounds will exhibit an IC₅₀ with respect to miRNA of not more than about 200 nM, as measured in the miRNA assay described herein.

Certain compounds disclosed herein are effective at reducing the level of mature miR-10b in cell culture. In some embodiments, compounds reduce the level of mature miR-10b by 20% or more. In some embodiments, compounds reduce the level of mature miR-10b by 30% or more. In some embodiments, compounds reduce the level of mature miR-10b by 40% or more. In some embodiments, compounds reduce the level of mature miR-10b by 50% or more. In certain embodiments, the cell culture is the AGS gastric cell line. In certain embodiments, the cell culture is the AsPC1 pancreatic cell line. In certain embodiments, the cell culture is the LN229 brain cancer cell line. In certain embodiments, the cell culture is the U251 brain cancer cell line.

Certain compounds disclosed herein are effective at reducing proliferation. In some embodiments, compounds reduce proliferation by 20% or more. In some embodiments, compounds reduce proliferation by 30% or more. In some embodiments, compounds reduce proliferation by 40% or more. In some embodiments, compounds reduce proliferation by 50% or more. In certain embodiments, the cell culture is the AGS gastric cell line. In certain embodiments, the cell culture is the AsPC1 pancreatic cell line. In certain embodiments, the cell culture is the LN229 brain cancer cell line. In certain embodiments, the cell culture is the U251 brain cancer cell line.

Certain compounds disclosed herein selectively reduce the level of mature miR-10b over other micro-RNAs. In certain embodiments, the compounds are selective for miR-10b over miR-16. In certain embodiments, the compounds are selective for miR-10b over miR-28. In certain embodiments, the compounds are selective for miR-10b over miR-182. In certain embodiments, the compounds are selective for miR-10b over miR-10a. In certain embodiments, the compounds are selective for miR-10b over miR-21. In certain embodiments, the compounds are selective for miR-10b over miR-155. In certain embodiments, the cell culture is the AGS gastric cell line. In certain embodiments, the cell culture is the AsPC1 pancreatic cell line. In certain embodiments, the cell culture is the LN229 brain cancer cell line. In certain embodiments, the cell culture is the U251 brain cancer cell line.

Certain compounds disclosed herein are effective at upregulating HOXD10. In certain embodiments, the compounds upregulate HOXD10 by 50% over negative control. In certain embodiments, the compounds upregulate HOXD10 by 100% over negative control. In certain embodiments, the compounds upregulate HOXD10 by 150% over negative control.

The phrase “therapeutically effective” is intended to qualify the amount of a compound as disclosed herein, or a salt or tautomer thereof, used in the treatment of a disease or disorder or on the effecting of a clinical endpoint.

The term “therapeutically acceptable” refers to those compounds (or salts or tautomers thereof) which are suitable for use in contact with the tissues of patients without undue toxicity, irritation, and allergic response, are commensurate with a reasonable benefit/risk ratio, and are effective for their intended use.

As used herein, treatment of a disease refers to a method that improves the health of a patient or patients having said disease. In some embodiments, treatment of a disease comprises improving the morbidity of a patient or patients having said disease. In some embodiments, treatment of a disease comprises improving the mortality of a patient or patients having said disease. In some embodiments, treatment of a disease comprises improving the progression-free survival of a patient or patients having said disease. In some embodiments, treatment of a disease comprises ameliorating the effects of said disease in a patient or patients. In some embodiments, treatment of a disease comprises improving the quality of life in a patient or patients having said disease. As used herein, treatment of a disease does not embrace either prevention or prophylaxis of said disease. Stated differently, as used herein, treatment of a disease is intended to refer to methods directed to a patient or patients who have contracted said disease.

The term “patient” is generally synonymous with the term “subject” and includes all mammals including humans Examples of patients include humans, livestock such as cows, goats, sheep, pigs, and rabbits, and companion animals such as dogs, cats, rabbits, and horses. Preferably, the patient is a human.

The compounds disclosed herein can exist as salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

Salts of the compounds as disclosed herein can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, also provided are sodium, potassium, magnesium, and calcium salts of the compounds disclosed herein, and the like.

Basic addition salts of the compounds as disclosed herein can be prepared during the final isolation and purification of the compounds by reacting a carboxy group with a suitable base such as the hydroxide, carbonate, or bicarbonate of a metal cation or with ammonia or an organic primary, secondary, or tertiary amine The cations of salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine Other representative organic amines useful for the formation of base addition salts include ethylenediamine, ethanolamine, diethanolamine, piperidine, and piperazine.

### Pharmaceutical Compositions

While it may be possible for the compounds of the subject disclosure to be administered as the raw chemical, it is also possible to present them as a pharmaceutical formulation. Accordingly, provided herein are pharmaceutical formulations which comprise one or more of certain compounds disclosed herein, or one or more salts, or tautomers thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject disclosure or a salt or tautomer thereof with the carrier which constitutes one or more accessory ingredients. In general, the formulations are prepared by uniformly and intimately bringing into association the compound as disclosed herein, or salt or tautomer thereof, with liquid carriers or finely divided solid carriers or both and then, if necessary, shaping the product into the desired formulation.

### Oral Administration

The compounds of the present disclosure may be administered orally, including swallowing, so the compound enters the gastrointestinal tract, or is absorbed into the blood stream directly from the mouth, including sublingual or buccal administration.

Suitable compositions for oral administration include solid formulations such as tablets, pills, cachets, lozenges and hard or soft capsules, which can contain liquids, gels, powders, or granules, solutions or suspensions in an aqueous liquid or a non-aqueous liquid, or as an oil-in-water liquid emulsion or a water-in-oil liquid emulsion. A compound as disclosed herein, or a salt or tautomer thereof, may also be presented as a bolus, electuary or paste.

In a tablet or capsule dosage form the amount of drug present may be from about 0.05% to about 95% by weight, more typically from about 2% to about 50% by weight of the dosage form.

In addition, tablets or capsules may contain a disintegrant, comprising from about 0.5% to about 35% by weight, more typically from about 2% to about 25% of the dosage form. Examples of disintegrants include methyl cellulose, sodium or calcium carboxymethyl cellulose, croscarmellose sodium, polyvinylpyrrolidone, hydroxypropyl cellulose, starch and the like.

Suitable binders, for use in a tablet, include gelatin, polyethylene glycol, sugars, gums, starch, hydroxypropyl cellulose and the like. Suitable diluents, for use in a tablet, include mannitol, xylitol, lactose, dextrose, sucrose, sorbitol and starch.

Suitable surface active agents and glidants, for use in a tablet or capsule, may be present in amounts from about 0.1% to about 3% by weight, and include polysorbate 80, sodium dodecyl sulfate, talc and silicon dioxide.

Suitable lubricants, for use in a tablet or capsule, may be present in amounts from about 0.1% to about 5% by weight, and include calcium, zinc or magnesium stearate, sodium stearyl fumarate and the like.

Tablets may be made by compression or molding, optionally with one or more accessory ingredients. Compressed tablets may be prepared by compressing in a suitable machine a compound as disclosed herein, or a salt or tautomer thereof, in a free-flowing form such as a powder or granules, optionally mixed with binders, inert diluents, or lubricating, surface active or dispersing agents. Molded tablets may be made by molding in a suitable machine a mixture of the powdered compound moistened with a liquid diluent. Dyes or pigments may be added to tablets for identification or to characterize different combinations of doses of a compound as disclosed herein, or salt or tautomer thereof,.

Liquid formulations can include emulsions, solutions, syrups, elixirs and suspensions, which can be used in soft or hard capsules. Such formulations may include a pharmaceutically acceptable carrier, for example, water, ethanol, polyethylene glycol, cellulose, or an oil. The formulation may also include one or more emulsifying agents and/or suspending agents.

Compositions for oral administration may be formulated as immediate or modified release, including delayed or sustained release, optionally with enteric coating.

In another embodiment, a pharmaceutical composition comprises a therapeutically effective amount of a compound as disclosed herein, or a salt or tautomer thereof, and a pharmaceutically acceptable carrier.

Pharmaceutical preparations of compounds as disclosed herein, or salts or tautomers thereof, which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the compound as disclosed herein, or salt or tautomer thereof, in a free-flowing form such as a powder or granules, optionally mixed with binders, inert diluents, or lubricating, surface active or dispersing agents. Molded tablets may be made by molding in a suitable machine a mixture of the compound as disclosed herein, or salt or tautomer thereof, in powdered form, moistened with an inert liquid diluent. The tablets may optionally be coated or scored and may be formulated so as to provide slow or controlled release of the compound as disclosed herein, or salt or tautomer thereof. All formulations for oral administration should be in dosages suitable for such administration. The push-fit capsules can contain the compound as disclosed herein, or salt or tautomer thereof, in admixture with filler such as lactose, binders such as starches, and/or lubricants such as talc or magnesium stearate and, optionally, stabilizers. In soft capsules, the compound as disclosed herein, or salt or tautomer thereof, may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of doses of the compound as disclosed herein, or salt or tautomer thereof.

### Parenteral Administration

Compounds of the present disclosure, or salts or tautomers thereof, may be administered directly into the blood stream, muscle, or internal organs by injection, e.g., by bolus injection or continuous infusion. Suitable means for parenteral administration include intravenous, intra-muscular, subcutaneous intraarterial, intraperitoneal, intrathecal, intracranial, and the like. Suitable devices for parenteral administration include injectors (including needle and needle-free injectors) and infusion methods. The formulations may be presented in unit-dose or multi-dose containers, for example sealed ampoules and vials.

Most parenteral formulations are aqueous solutions containing excipients, including salts, buffering, suspending, stabilizing and/or dispersing agents, antioxidants, bacteriostats, preservatives, and solutes which render the formulation isotonic with the blood of the intended recipient, and carbohydrates.

Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions. Compositions for parenteral administration may be formulated as immediate or modified release, including delayed or sustained release. Compounds as disclosed herein, or salts or tautomers thereof, may also be formulated as depot preparations. Such long acting formulations may be administered by implantation (for example subcutaneously or intramuscularly) or by intramuscular injection. Thus, for example, the compounds as disclosed herein, or salts or tautomers thereof, may be formulated with suitable polymeric or hydrophobic materials (for example as an emulsion in an acceptable oil) or ion exchange resins, or as sparingly soluble derivatives, for example, as a sparingly soluble salt.

The compounds as disclosed herein, or salts or tautomers thereof, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of a compound as disclosed herein, or a salt or tautomer thereof, which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compound as disclosed herein, or salt or tautomer thereof, to allow for the preparation of highly concentrated solutions.

In addition to the formulations described previously, compounds as disclosed herein, or salts or tautomer thereof, may also be formulated as a depot preparation. Such long acting formulations may be administered by implantation (for example subcutaneously or intramuscularly) or by intramuscular injection. Thus, for example, compounds as disclosed herein, or salts or tautomers thereof, may be formulated with suitable polymeric or hydrophobic materials (for example as an emulsion in an acceptable oil) or ion exchange resins, or as sparingly soluble derivatives, for example, as a sparingly soluble salt.

Topical Administration

Compounds of the present disclosure, or salts or tautomers thereof, may be administered topically (for example to the skin, mucous membranes, ear, nose, or eye) or transdermally. Formulations for topical administration can include, but are not limited to, lotions, solutions, creams, gels, hydrogels, ointments, foams, implants, patches and the like. Carriers that are pharmaceutically acceptable for topical administration formulations can include water, alcohol, mineral oil, glycerin, polyethylene glycol and the like. Topical administration can also be performed by, for example, electroporation, iontophoresis, phonophoresis and the like.

Typically, a formulation of a compound as disclosed herein, or a salt or tautomer thereof, for topical administration may comprise from 0.001% to 10% w/w (by weight) of the the compound as disclosed herein, or salt or tautomer thereof. In certain embodiments, the compound as disclosed herein, or salt or tautomer thereof, may comprise as much as 10% w/w; less than 5% w/w; from 2% w/w to 5% w/w; or from 0.1% to 1% w/w of the formulation.

Compositions for topical administration may be formulated as immediate or modified release, including delayed or sustained release.

Certain compounds disclosed herein, or salts or tautomers thereof, may be administered topically, that is by non-systemic administration. This includes the application of a compound disclosed herein, or a salt or tautomer thereof, externally to the epidermis or the buccal cavity and the instillation of such a compound, or salt or tautomer thereof, into the ear, eye and nose, such that the compound, or salt or tautomer thereof, does not significantly enter the blood stream. In contrast, systemic administration refers to oral, intravenous, intraperitoneal and intramuscular administration.

Formulations of compounds as disclosed herein, or salts or tautomers thereof, suitable for topical administration include liquid or semi-liquid preparations suitable for penetration through the skin to the site of inflammation such as gels, liniments, lotions, creams, ointments or pastes, and drops suitable for administration to the eye, ear or nose. The formulation for topical administration may comprise, for example, from 0.001% to 10% w/w (by weight) of the compound as disclosed herein, or salt or tautomer thereof. In certain embodiments, the formulation may comprise as much as 10% w/w of the compound as disclosed herein, or salt or tautomer thereof. In other embodiments, it may comprise less than 5% w/w of the compound as disclosed herein, or salt or tautomer thereof,. In certain embodiments, the formulation may comprise from 2% w/w to 5% w/w of the compound as disclosed herein, or salt or tautomer thereof. In other embodiments, it may comprise from 0.1% to 1% w/w of the compound as disclosed herein, or salt or tautomer thereof.

### Rectal, Buccal, and Sublingual Administration

Suppositories for rectal administration of the compounds of the present disclosure, or salts or tautomers thereof, can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

For buccal or sublingual administration, the compositions may take the form of tablets, lozenges, pastilles, or gels formulated in conventional manner Such compositions may comprise a compound as disclosed herein, or a salt or tautomer thereof, in a flavored basis such as sucrose and acacia or tragacanth.

The compounds, or salts or tautomers thereof, may also be formulated in rectal compositions such as suppositories or retention enemas, e.g., containing conventional suppository bases such as cocoa butter, polyethylene glycol, or other glycerides.

### Administration by Inhalation

For administration by inhalation, compounds, or salts or tautomers thereof, may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds, or salts or tautomers thereof, according to the disclosure may take the form of a dry powder composition, for example a powder mix of the compound, or a salt or tautomer thereof, and a suitable powder base such as lactose or starch. The powder composition may be presented in unit dosage form, in for example, capsules, cartridges, gelatin or blister packs from which the powder may be administered with the aid of an inhalator or insufflator.

Other carrier materials and modes of administration known in the pharmaceutical art may also be used. Pharmaceutical compositions of the disclosure may be prepared by any of the well-known techniques of pharmacy, such as effective formulation and administration procedures. Preferred unit dosage formulations are those containing an effective dose, as herein below recited, or an appropriate fraction thereof, of a compound as disclosed herein, or a salt or tautomer thereof.

It should be understood that in addition to the ingredients particularly mentioned above, the formulations described above may include other agents conventional in the art having regard to the type of formulation in question, for example those suitable for oral administration may include flavoring agents.

Compounds, or salts or tautomers thereof, may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds, or salts or tautomers thereof, which is effective at such dosage or as a multiple of the same, for instance, units containing 5 mg to 500 mg, usually around 10 mg to 200 mg.

The amount of a compound as disclosed herein, or a salt or tautomer thereof, that may be combined with the carrier materials to produce a single dosage form will vary depending upon the host treated and the particular mode of administration.

The compounds, or salts or tautomers thereof, can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. In addition, the route of administration may vary depending on the condition and its severity. The above considerations concerning effective formulations and administration procedures are well known in the art and are described in standard textbooks.

Preferred unit dosage formulations are those containing an effective dose, as herein below recited, or an appropriate fraction thereof, of a compound as disclosed herein, or a salt or tautomer thereof,.

It should be understood that in addition to the ingredients particularly mentioned above, the formulations described above may include other agents conventional in the art having regard to the type of formulation in question, for example those suitable for oral administration may include flavoring agents.

### Combinations and Combination Therapy

In certain instances, it may be appropriate to administer at least one of the compounds described herein (or a salt or tautomer thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating miRNA-mediated disorders in a human or animal subject in need of such treatment comprising administering to said subject an amount of a compound disclosed herein, or a salt or tautomer thereof, effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of miRNA-mediated disorders.

Cancers to be treated by the methods disclosed herein include colon cancer, breast cancer, ovarian cancer, lung cancer and prostate cancer; cancers of the oral cavity and pharynx (lip, tongue, mouth, larynx, pharynx), esophagus, stomach, small intestine, large intestine, colon, rectum, liver and biliary passages; pancreas, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; and thyroid and other endocrine glands. The term “cancer” also encompasses cancers that do not necessarily form solid tumors, including Hodgkin's disease, non-Hodgkin's lymphomas, multiple myeloma and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML),) and lymphomas including lymphocytic, granulocytic and monocytic. Additional types of cancers which may be treated using the compounds and methods of the disclosure include, but are not limited to, adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumours, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor.

In certain embodiments, the compositions and methods disclosed herein are useful for preventing or reducing tumor invasion and tumor metastasis.

For use in cancer and neoplastic diseases a RIPK1 inhibitor may be optimally used together with one or more of the following non-limiting examples of anti-cancer agents:

1) inhibitors or modulators of a protein involved in one or more of the DNA damage repair (DDR) pathways such as:
a. PARP1/2, including, but not limited to: olaparib, niraparib, rucaparib;
b. checkpoint kinase 1 (CHK1), including, but not limited to: UCN-01,

AZD7762, PF477736, SCH900776, MK-8776, LY2603618, V158411, and EXEL-9844;
c. checkpoint kinase 2 (CHK2), including, but not limited to: PV1019, NSC 109555, and VRX0466617;
d. dual CHK1/CHK2, including, but not limited to: XL-844, AZD7762, and PF-473336;
e. WEE1, including, but not limited to: MK-1775 and PD0166285;
f. ATM, including, but not limited to KU-55933,
g. DNA-dependent protein kinase, including, but not limited to NU7441 and M3814; and
h. Additional proteins involved in DDR; po 2) Inhibitors or modulators of one or more immune checkpoints, including, but not limited to:
a. PD-1 inhibitors such as nivolumab (OPDIVO), pembrolizumab (KEYTRUDA), pidilizumab (CT-011), and AMP-224 (AMPLIMMUNE);
b. PD-L1 inhibitors such as Atezolizumab (TECENTRIQ), Avelumab (Bavencio), Durvalumab (Imfinzi), MPDL3280A (Tecentriq), BMS-936559, and MEDI4736;
c. anti-CTLA-4 antibodies such as ipilimumab (YERVOY) and CP-675,206 (TREMELIMUMAB);
d. inhibitors of T-cell immunoglobulin and mucin domain 3 (Tim-3);
e. inhibitors of V-domain Ig suppressor of T cell activation (Vista);
f. inhibitors of band T lymphocyte attenuator (BTLA);
g. inhibitors of lymphocyte activation gene 3 (LAG3); and
h. inhibitors of T cell immunoglobulin and immunoreceptor tyrosine-based inhibitory motif domain (TIGIT);

3) telomerase inhibitors or telomeric DNA binding compounds;

4) alkylating agents, including, but not limited to: chlorambucil (LEUKERAN), oxaliplatin (ELOXATIN), streptozocin (ZANOSAR), dacarbazine, ifosfamide, lomustine (CCNU), procarbazine (MATULAN), temozolomide (TEMODAR), and thiotepa;

5) DNA crosslinking agents, including, but not limited to: carmustine, chlorambucil (LEUKERAN), carboplatin (PARAPLATIN), cisplatin (PLATIN), busulfan (MYLERAN), melphalan (ALKERAN), mitomycin (MITOSOL), and cyclophosphamide (ENDOXAN);

6) anti-metabolites, including, but not limited to: cladribine (LEUSTATIN), cytarbine,

(ARA-C), mercaptopurine (PURINETHOL), thioguanine, pentostatin (NIPENT), cytosine arabinoside (cytarabine, ARA-C), gemcitabine (GEMZAR), fluorouracil (5-FU, CARAC), capecitabine (XELODA), leucovorin (FUSILEV), methotrexate (RHEUMATREX), and raltitrexed;

7) antimitotic, which are often plant alkaloids and terpenoids, or derivatives thereof including but limited to: taxanes such as docetaxel (TAXITERE), paclitaxel (ABRAXANE, TAXOL), vinca alkaloids such as vincristine (ONCOVIN), vinblastine, vindesine, and vinorelbine (NAVELBINE);

8) topoisomerase inhibitors, including, but not limited to: amacrine, camptothecin (CTP), genistein, irinotecan (CAMPTOSAR), topotecan (HYCAMTIN), doxorubicin (ADRIAMYCIN), daunorubicin (CERUBIDINE), epirubicin (ELLENCE), ICRF-193, teniposide (VUMON), mitoxantrone (NOVANTRONE), and etoposide (EPOSIN);

9) DNA replication inhibitors, including, but not limited to: fludarabine (FLUDARA), aphidicolin, ganciclovir, and cidofovir;

10) ribonucleoside diphosphate reductase inhibitors, including, but not limited to:

hydroxyurea;

11) transcription inhibitors, including, but not limited to: actinomycin D (dactinomycin, COSMEGEN) and plicamycin (mithramycin);

12) DNA cleaving agents, including, but not limited to: bleomycin (BLENOXANE), idarubicin,

13) cytotoxic antibiotics, including, but not limited to: actinomycin D (dactinomycin, COSMEGEN),

14) aromatase inhibitors, including, but not limited to aminoglutethimide, anastrozole (ARIMIDEX), letrozole (FEMARA), vorozole (RIVIZOR), and exemestane (AROMASIN);

15) angiogenesis inhibitors, including, but not limited to: genistein, sunitinib (SUTENT), and bevacizumab (AVASTIN);

16) anti-steroids and anti-androgens, including, but not limited to aminoglutethimide (CYTADREN), bicalutamide (CAS ODEX), cyproterone, flutamide (EULEXIN), nilutamide(NILANDRON);

17) tyrosine kinase inhibitors, including, but not limited to imatinib (GLEEVEC), erlotinib (TARCEVA), lapatininb (TYKERB), sorafenib (NEXAVAR), and axitinib (INLYTA);

18) mTOR inhibitors, including, but not limited to: everolimus, temsirolimus (TORISEL), and sirolimus;

19) monoclonal antibodies, including, but not limited to: trastuzumab (HERCEPTIN) and rituximab (RITUXAN);

20) apoptosis inducers such as cordycepin;

21) protein synthesis inhibitors, including, but not limited to: clindamycin, chloramphenicol, streptomycin, anisomycin, and cycloheximide;

22) antidiabetics, including, but not limited to: metformin and phenformin;

23) antibiotics, including, but not limited to:
a. tetracyclines, including, but not limited to: doxycycline;
b. erythromycins, including, but not limited to: azithromycin;
c. glycylglycines, including, but not limited to: tigecycline;
d. antiphrastic, including, but not limited to: pyrvinium pamoate;
e. beta-lactams, including, but not limited to the penicillins and cephalosporins;
f. anthracycline antibiotics, including, but not limited to: daunorubicin and doxorubicin;
g. other antibiotics, including, but not limited to: chloramphenicol, mitomycin C, and actinomycin;

24) antibody therapeutic agents, including, but not limited to: muromonab-CD3, infliximab (REMICADE), adalimumab (HUMIRA), omalizumab (XOLAIR), daclizumab (ZENAPAX), rituximab (RITUXAN), ibritumomab (ZEVALIN), tositumomab (BEXXAR), cetuximab (ERBITUX), trastuzumab (HERCEPTIN), ADCETRIS, alemtuzumab (CAMPATH-1H), Lym-1 (ONCOLYM), ipilimumab (YERVOY), vitaxin, bevacizumab (AVASTIN), and abciximab (REOPRO); and 25) other agents, such as Bacillus Calmette-Guérin (B-C-G) vaccine; buserelin

(ETILAMIDE); chloroquine (ARALEN); clodronate, pamidronate, and other bisphosphonates; colchicine; demethoxyviridin; dichloroacetate; estramustine; filgrastim (NEUPOGEN); fludrocortisone (FLORINEF); goserelin (ZOLADEX); interferon; leucovorin; leuprolide (LUPRON); levamisole; lonidamine; mesna; metformin; mitotane (o,p′-DDD, LYSODREN); nocodazole; octreotide (SANDOSTATIN); perifosine; porfimer (particularly in combination with photo- and radiotherapy); suramin; tamoxifen; titanocene dichloride; tretinoin; anabolic steroids such as fluoxymesterone (HALOTESTIN); estrogens such as estradiol, diethylstilbestrol (DES), and dienestrol; progestins such as medroxyprogesterone acetate (MPA) and megestrol; and testosterone.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### Compound Synthesis

Compounds of the present disclosure, or salts or tautomers thereof, can be prepared using methods illustrated in general synthetic schemes and experimental procedures detailed below. General synthetic schemes and experimental procedures are presented for purposes of illustration and are not intended to be limiting. Starting materials used to prepare compounds of the present disclosure are commercially available or can be prepared using routine methods known in the art.

### List of Abbreviations

Ac₂O=acetic anhydride; AcC;=acetyl chloride; AcOH=acetic acid; AIBN=azobisisobutyronitrile; aq.=aqueous; Bu₃SnH=tributyltin hydride; CD₃OD=deuterated methanol; CDCl₃=deuterated chloroform; CDI=1,1′-Carbonyldiimidazole; DBU=1,8-diazabicyclo[5.4.0]undec-7-ene; DCM=dichloromethane; DEAD=diethyl azodicarboxylate; DIBAL-H=di-iso-butyl aluminium hydride; DIEA=DIPEA=N,N-diisopropylethylamine; DMAP=4-dimethylaminopyridine; DMF=N,N-dimethylformamide; DMSO-d₆=deuterated dimethyl sulfoxide; DMSO=dimethyl sulfoxide; DPPA=diphenylphosphoryl azide; EDC.HCl=EDCI.HCl=1-ethyl-3-(3-dimethylaminopropyl)carbodiimide hydrochloride; Et₂O=diethyl ether; EtOAc=ethyl acetate; EtOH=ethanol; h=hour; HATU=2-(1H-7-azabenzotriazol-1-yl)-1,1,3,3-tetramethyl uronium hexafluorophosphate methanaminium; HMDS=hexamethyldisilazane; HOBT=1-hydroxybenzotriazole; i-PrOH=isopropanol; LAH=lithium aluminium hydride; LiHMDS=Lithium bis(trimethylsilyl)amide; MeCN=acetonitrile; MeOH=methanol; MP-carbonate resin=macroporous triethylammonium methylpolystyrene carbonate resin; MsCl=mesyl chloride; MTBE=methyl tertiary butyl ether; MTS=3-(4,5-dimethylthiazol-2-yl)-5-(3-carboxymethoxyphenyl)-2-(4-sulfophenyl)-2H-tetrazolium, inner salt; MTT=3-(4,5-dimethyl-2-thiazolyl)-2,5-diphenyl-2H-tetrazolium bromide; MW=microwave irradiation; n-BuLi=n-butyllithium; NaHMDS=Sodium bis(trimethylsilyl)amide; NaOMe=sodium methoxide; NaOtBu=sodium t-butoxide; NBS=N-bromosuccinimide; NCS=N-chloro-succinimide; NMP=N-Methyl-2-pyrrolidone; OD=optical density; Pd(Ph₃)₄=tetrakis-(triphenylphosphine)palladium(0); Pd₂(dba)₃=tris(dibenzylideneacetone)dipalladium(0); PdCl₂(PPh₃)₂=bis(triphenylphosphine)palladium(II) dichloride; PG=protecting group; prep-HPLC=preparative high-performance liquid chromatography; PyBop=(benzotriazol-1-yloxy)tripyrrolidinophosphonium hexafluorophosphate; Pyr=pyridine; RT=room temperature; RuPhos=2-dicyclohexylphosphino-2′,6′-diisopropoxybiphenyl; sat.=saturated; ss=saturated solution; t-BuOH=tert-butanol; T3P=Propylphosphonic Anhydride; TBS=TBDMS=tert-butyldimethylsilyl; TBSCl=TBDMSCl=tert-butyldimethylchlorosilane; TEA=Et₃N=triethylamine; TFA=trifluoroacetic acid; TFAA=trifluoroacetic anhydride; THF=tetrahydrofuran; Tot=toluene; TsCl=tosyl chloride; WST-8=5-(2,4-disulfophenyl)-3-(2-methoxy-4-nitrophenyl)-2-(4-nitrophenyl)-2H- tetrazolium, inner salt, sodium salt, XPhos=2-dicyclohexylphosphino-2′,4′,6′-triisopropylbiphenyl.

### General Synthetic Methods for Preparing Compounds

The following schemes can be used to practice the present disclosure.

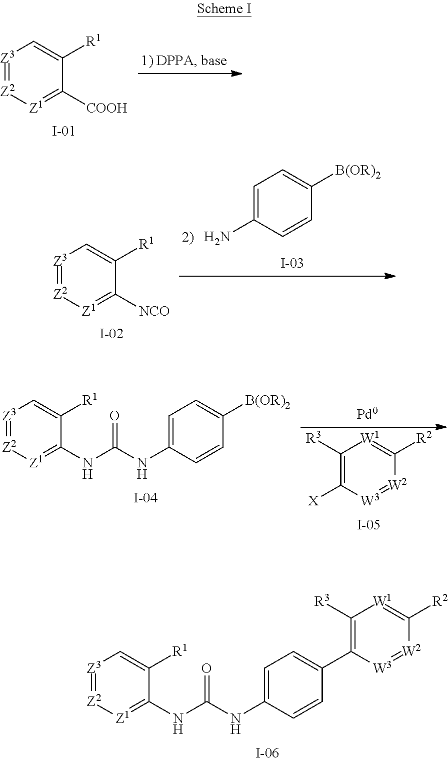

Certain examples can be synthesized using the following general synthetic procedure set forth in Scheme I. Arenecarboxylic acid I-01 (Z¹, Z², Z³, and R¹ are as described herein) is converted to the isocyanate I-02 by treatment with diphenylphosphoryl azide in the presence of base. Alternative methods to produce the intermediate acyl azide (not shown), including but not limited to treatment of the corresponding acid chloride with an azide salt, are known in the art. The isocyanate I-02 may be isolated, or may be converted directly to urea I-04 by treatment with substituted aniline I-03 (R is chosen from H and alkyl). Transition metal coupling of an aryl halide I-05 (W¹, W², W³, R², and R³ are as described herein, and X is chosen from Br and I) at the boronic acid functionality of 1-04 gives the biaryl moiety of product I-06.

Referring to Scheme I, in a first step a mixture of arenecarboxylic acid I-01, DPPA, and an initial amine base in an aprotic solvent is heated for a period of time between 1 h and 4 h. In some embodiments, the aprotic solvent is a nonpolar solvent such as hexane, benzene, or toluene. In some embodiments, about 1 equivalent of DPPA relative to I-01 is used. In some embodiments, about 2 equivalents of initial amine base relative to I-01 is used. In some embodiments, the initial amine base is TEA. In some embodiments, in the first step, the mixture is heated at about 90° C. In some embodiments, in the first step, the mixture is heated for a period of about 2 h. In some embodiments, compound I-02 is obtained after work-up of the reaction mixture. In some embodiments, in a second step, compound I-02, I-03 and additional amine base are combined in a suitable solvent after the first step, and the mixture is heated for a period of time between 12 h and 24 h. In some embodiments, compound I-02 is not isolated. In some embodiments, in a second step, I-03 and additional amine base is added to the solution after the first step, and the mixture is heated for a period of time between 12 h and 24 h. Compound I -04 is obtained after work-up of the reaction mixture. In some embodiments, in the second step, the mixture is heated for about 16 h. In some embodiments, in the second step, the mixture is heated at about 90° C. In some embodiments, about 1 equivalent I-03 of relative to I-01 is added. In some embodiments, about 2 equivalent of additional amine base relative to I-01 is used. In some embodiments, the initial amine base and the additional amine base are identical. In some embodiments, the initial amine base and the additional amine base are TEA.

Referring to Scheme I, a mixture of urea I04, aryl halide I-05 (X═Br, I), a Pd(0) catalyst, and a base in a suitable solvent or solvent mixture is heated for a period of time between 12 h and 24 h. Compound I-06 is obtained after work-up of the reaction mixture. In some embodiments, between 1.1 and 1.3 equivalents of I-05, relative to I-04, is used. In some embodiments, the Pd(0) catalyst is a tetra(phosphine)Pd(0) complex. In some embodiments, the Pd(0) catalyst is Pd(PPh₃)₄. In some embodiments, about 0.05 equivalents of the Pd(0) catalyst, relative to I-04, is used. In some embodiments, the base is an inorganic base. In some embodiments, the base is an inorganic carbonate base. In some embodiments, the base is Na₂CO₃. In some embodiments, between 2.0 and 2.5 equivalents of base, relative to I-04, is used. In some embodiments, a solvent mixture is used. In some embodiments, a solvent mixture comprising a hydrophobic organic solvent, an alcoholic solvent, and water is used. In some embodiments, the solvent mixture comprises toluene. In some embodiments, the solvent mixture comprises EtOH. In some embodiments, the mixture is heated at about 90° C. In some embodiments, the mixture is heated for about 16 h.

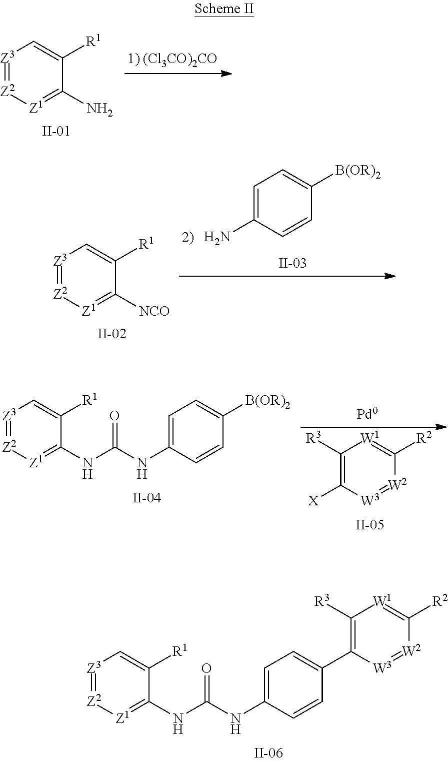

Certain examples can be synthesized using the following general synthetic procedure set forth in Scheme II. Arylamine II-01 (Z¹, Z², Z³, and R¹ are as described herein) is reacted with triphosgene to produce isocyanate II-02. The isocyanate II-02 may be isolated, or may be converted directly to urea II-04 by treatment with substituted aniline II-03 (R is chosen from H and alkyl). Transition metal coupling of an aryl halide II-05 (W¹, W², W³, R², and R³ are as described herein, and X is chosen from Br and I) at the boronic acid functionality of 11-04 gives the biaryl moiety of product II-06.

Referring to Scheme II, to a mixture of II-01 in a nonpolar solvent is added triphosgene. The mixture is refluxed for a period of time between 2 h and 6 h, then concentrated to provide isocyanate II-02. In certain embodiments, the nonpolar solvent is toluene. In certain embodiments, the mixture is refluxed for about 4 h. In certain embodiments, between 0.3 and 0.4 equiv of triphosgene, relative to II-01, is used. In certain embodiments, between 0.35 and 0.4 equiv of triphosgene, relative to II-01, is used.

Referring to Scheme II, a mixture of II-02 and II-03 in a polar solvent is refluxed for a period of time between 12 h and 24 h. After work-up of the reaction mixture, II-04 is obtained. In certain embodiments, II-04 is purified by silica column chromatography. In certain embodiments, between 1.0 and 1.2 equivalents of II-03, relative to II-01, is used. In certain embodiments, the polar solvent is an ethereal solvent. In certain embodiments, the ethereal solvent is chosen from THF, dioxane, and 1,2-dimethoxyethane. In certain embodiments, the mixture is refluxed for about 16 h.

Referring to Scheme II, a mixture of urea II-04, aryl halide II-05 (X═Br, I), a Pd(0) catalyst, and a base in a suitable solvent or solvent mixture is heated for a period of time between 12 h and 24 hr. Compound II-06 is obtained after work-up of the reaction mixture. In certain embodiments, between 1.0 and 1.2 equivalents of II-05, relative to II-04, is used. In some embodiments, the Pd(0) catalyst is a tetra(phosphine)Pd(0) complex. In some embodiments, the Pd(0) catalyst is Pd(PPh₃)₄. In some embodiments, the base is an inorganic base. In some embodiments, the base is an inorganic carbonate base. In some embodiments, the base is Na₂CO₃. In some embodiments, a solvent mixture is used. In some embodiments, a solvent mixture comprising a hydrophobic organic solvent, an alcoholic solvent, and water is used. In some embodiments, the solvent mixture comprises toluene. In some embodiments, the solvent mixture comprises EtOH.

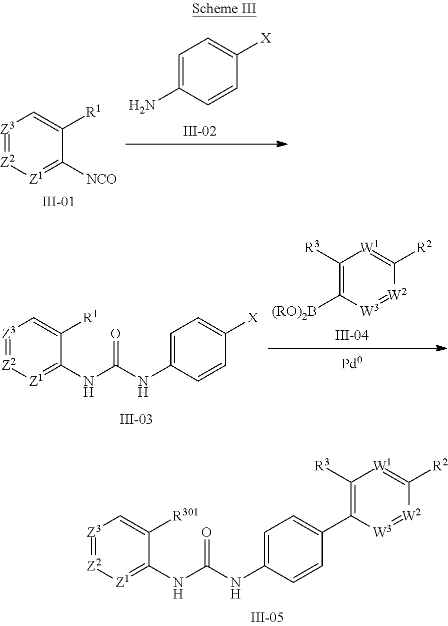

Certain examples can be synthesized using the following general synthetic procedure set forth in Scheme III. Aryl isocyanate III-01 (Z¹, Z², Z³, and R¹ are as described herein), produced by the method of Scheme I or Scheme II, or an alternative method, is reacted with halogenated aniline III-02 (X is chosen from Br and I) to give urea III-03. Transition metal coupling with an arylboronate III-04 (W¹, W², W³, R², and R³ are as described herein, and R is chosen from H and alkyl) gives the biaryl moiety of product III-05.

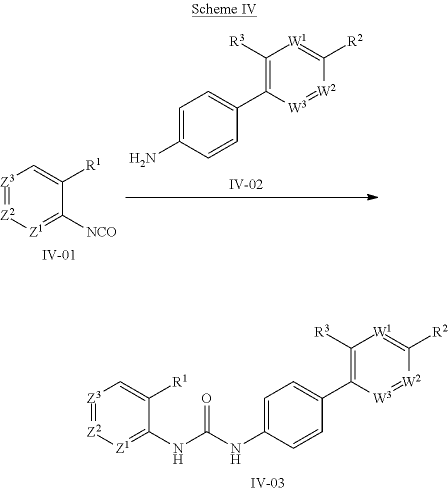

Certain examples can be synthesized using the following general synthetic procedure set forth in Scheme IV. Aryl isocyanate IV-01 (Z¹, Z², Z³, and R¹ are as described herein), produced by the method of Scheme I or Scheme II, or an alternative method, is reacted with biaryl amine IV-02 (W¹, W², W³, R², and R³ are as described herein) to give urea IV-03.

The disclosure is further illustrated by the following examples.

### EXAMPLE 1

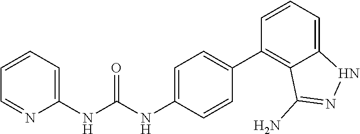 [SMILES: Nc1n[nH]c2cccc(-c3ccc(NC(=O)Nc4ccccn4)cc3)c12]

### 1-(4-(3-amino-1H-indazol-4-yl)phenyl)-3-(pyridin-2-yl)urea

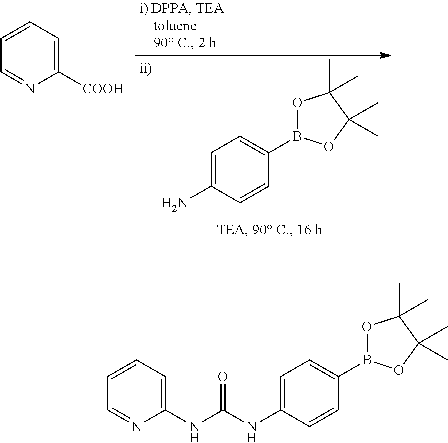 [SMILES: O=C(O)c1ccccn1 ; CC1(C)OB(c2ccc(N)cc2)OC1(C)C ; CC1(C)OB(c2ccc(NC(=O)Nc3ccccn3)cc2)OC1(C)C]

1-(Pyridin-2-yl)-3-(4-(4,4,5,5-tetramethyl-1,3,2-dioxaborolan-2-yl)pheny;)urea A mixture of picolinic acid (123 mg, 1 mmol), DPPA (274 mg, 1 mmol) and TEA (202 mg, 2 mmol) in 5 mL of toluene was heated at 90° C. for 2 h. 4-(4,4,5,5-Tetramethyl-1,3,2-dioxaborolan-2-yl)aniline (200 mg, 1 mmol) and TEA (202 mg, 2 mmol) was added and the mixture was heated at 90° C. for 16 h. The crude product was purified by column chromatography (EtOAc in PE, 50% to 100%) to afford the title compound (150 mg, 49%) as a yellow oil. MS (ES+) C₁₈H₂₂BN₃O₃ requires: 339, found: 340[M+H]⁺.

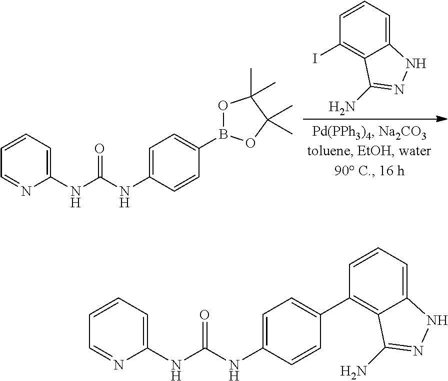 [SMILES: CC1(C)OB(c2ccc(NC(=O)Nc3ccccn3)cc2)OC1(C)C ; Nc1n[nH]c2cccc(I)c12 ; Nc1n[nH]c2cccc(-c3ccc(NC(=O)Nc4ccccn4)cc3)c12]

1-(4-(3-Amino-1H-indazol-4-yl)phenyl)-3-(pyridin-2-yl)urea A mixture of the product from the previous step (150 mg, 0.44 mmol), 4-iodo-1H-indazol-3-amine (138 mg, 0.53 mmol), Na₂CO₃ (94 mg, 0.88 mmol) and Pd(PPh₃)₄ (26 mg, 0.022 mmol) in toluene/EtOH/H₂O (2 mL/1 mL/1 mL) was stirred at 90° C. for 16 h under Ar. Water and EtOAc was added, and the resulting solid was removed by filtration to afford the title compound as a beige solid (85 mg, 56%).

MS (ES+) C₁₉H₁₆N₆O requires: 344, found: 345[M+H]⁺.

¹H NMR (500 MHz, DMSO) 67 11.73 (s, 1H), 10.70 (s, 1H), 9.54 (s, 1H), 8.31 (d, J=4.3 Hz, 1H), 7.77 (t, J=7.8 Hz, 1H), 7.68 (d, J=8.3 Hz, 2H), 7.51 (d, J=8.3 Hz, 1H), 7.43 (d, J=8.3 Hz, 2H), 7.31−7.22 (m, 2H), 7.06−6.99 (m, 1H), 6.80 (d, J=5.8 Hz, 1H), 4.34 (s, 2H).

### EXAMPLE 2

### 1-(2-fluoro-5-methylphenyl)-3-(4-(pyrimidin-5-yl)phenyl)urea

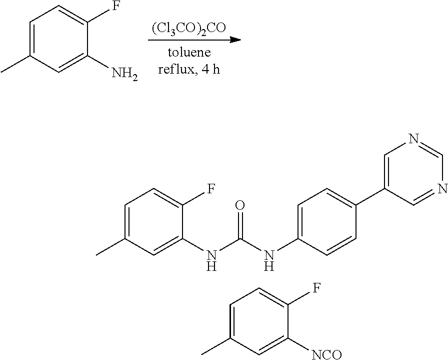

1-Fluoro-2-isocyanato-4-methylbenzene To a mixture of 2-fluoro-5-methylaniline (1.25 g, 10 mmol) in toluene (20 ml) was added triphosgene (1.09 g, 3.67 mmol), and the mixture was refluxed for 4 h. The mixture was concentrated and used directly for next step without further purification.

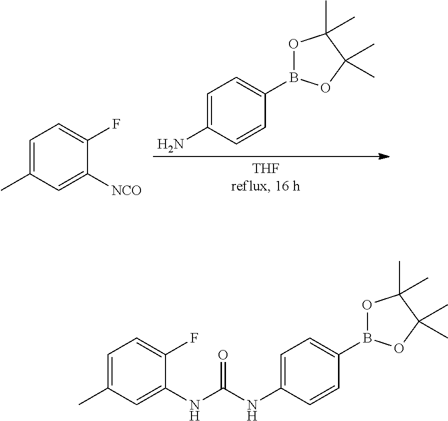

1-(2-Fluoro-5-methylphenyl)-3-(4-(4,4,5,5-tetramethyl-1,3,2-dioxaborolan-2-yl)pheny)urea A mixture of the product from the previous step (1g, crude) and 4-(4,4,5,5-tetramethyl-1,3,2-dioxaborolan-2-yl)aniline (1.45 g, 6.6 mmol) in THF (15 ml) was refluxed for 16 h. The mixture was concentrated and purified by silica column (EtOAc in PE =0 to 80%) to obtain the title compound as a white solid (1.5 g, 61%). MS (ES+) C₂₀H₂₄BFN₂O₃ requires: 370, found: 371[M+H]⁺.

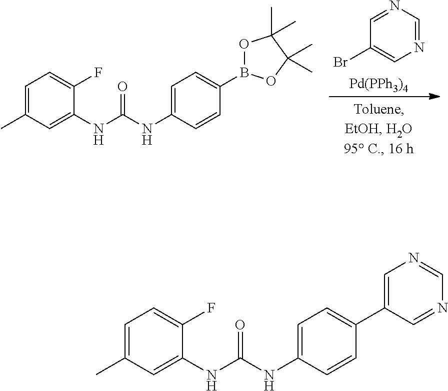

1-(2-Fluoro-5-methylphenyl)-3-(4-(pyrimidin-5-yl)phenyl)urea (2) A mixture of the product from the previous step (120 mg, 0.32 mmol), 5-bromopyrimidine (57 mg, 0.36 mmol) and Na₂CO₃ (74 mg, 0.7 mmol) and Pd(PPh₃)₄ (20 mg, 0.02 mmol) in toluene/EtOH/H₂O (2 mL/1 mL/1 mL) was stirred at 95° C. for 16 h under Ar. The mixture was concentrated and purified by prep-HPLC (NH₄HCO₃) to afford the title compound as a white solid (47 mg, 44%).

MS(ES+): C₁₈H₁₅FN₄O requires: 322, found: 323 [M+H]⁺.

¹H NMR (500 MHz, DMSO) δ 9.28 (s, 1H), 9.14 (d, J=6.5 Hz, 3H), 8.56 (d, J=1.7 Hz, 1H), 8.00 (d, J=6.8 Hz, 1H), 7.78 (d, J=8.6 Hz, 2H), 7.63 (d, J=8.6 Hz, 2H), 7.12 (dd, J=11.3, 8.4 Hz, 1H), 6.91−6.77 (m, 1H), 2.29 (s, 3H).

The following compounds were also synthesized by using methods similar to those used for the above compounds.

TABLE 1
Example compounds.

| Ex. | | | Ex. |
|---|---|---|---|
| No. | Structure | IUPAC Name | Method |
| 3 | 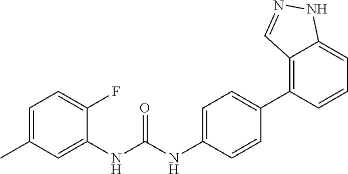 [SMILES: Cc1ccc(F)c(NC(=O)Nc2ccc(-c3cccc4[nH]ncc34)cc2)c1] | 1-(4-(1H-indazol-4- yl)phenyl)-3-(2- fluoro-5- methylphenyl)urea | |
| 4 | 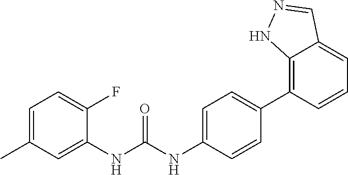 [SMILES: Cc1ccc(F)c(NC(=O)Nc2ccc(-c3cccc4cn[nH]c34)cc2)c1] | 1-(4-(1H-indazol-7- yl)phenyl)-3-(2- fluoro-5-methyl- phenyl)urea | 2 |
| 5 | 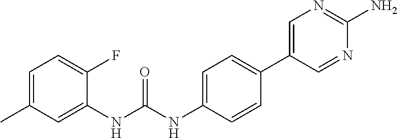 [SMILES: Cc1ccc(F)c(NC(=O)Nc2ccc(-c3cnc(N)nc3)cc2)c1] | 1-(4-(2-amino- pyrimidin-5- yl)phenyl)-3-(2- fluoro-5-methyl- phenyl)urea | 2 |
| 6 | 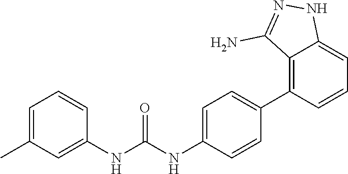 [SMILES: Cc1cccc(NC(=O)Nc2ccc(-c3cccc4[nH]nc(N)c34)cc2)c1] | 1-(4-(3-amino-1H- indazol-4- yl)phenyl)-3-(o- tolyl)urea | |
| 7 | 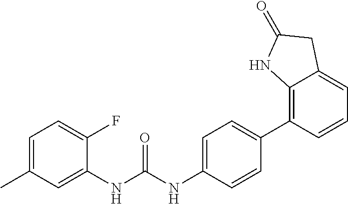 [SMILES: Cc1ccc(F)c(NC(=O)Nc2ccc(-c3cccc4c3NC(=O)C4)cc2)c1] | 1-(2-fluoro-5- methylphenyl)-3- (4-(2-oxoindolin-7- yl)phenyl)urea | |
| 8 | 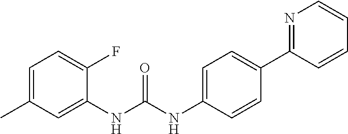 [SMILES: Cc1ccc(F)c(NC(=O)Nc2ccc(-c3ccccn3)cc2)c1] | 1-(2-fluoro-5- methylphenyl)-3- (4-(pyridin-2- yl)phenyl)urea | |
| 9 | 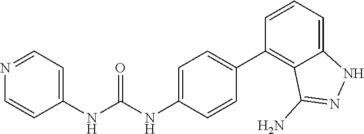 [SMILES: Nc1n[nH]c2cccc(-c3ccc(NC(=O)Nc4ccncc4)cc3)c12] | 1-(4-(3-amino-1H- indazol-4- yl)phenyl)-3- (pyridin-4-yl)urea | 1 |

TABLE 2
Characterization of example compounds.

| Ex. | | Exact | MS(ES+) | |
|---|---|---|---|---|
| No. | Formula | Mass | [M + H]⁺ | ¹H NMR (500 MHz, DMSO-d₆) |
| 3 | C₂₁H₁₇FN₄O | 360 | | |
| 4 | C₂₁H₁₇FN₄O | 360 | 361 | δ 13.17 (s, 1H), 9.25 (s, 1H), 8.55 (d, J = |
| | | | | 1.9 Hz, 1H), 8.17 (s, 1H), 8.03 (d, J = 7.7 |
| | | | | Hz, 1H), 7.74 (d, J = 8.0 Hz, 1H), 7.64 |
| | | | | (q, J = 8.6 Hz, 4H), 7.37 (t, J = 13.3 Hz, |
| | | | | 1H), 7.22-7.00 (m, 2H), 6.86-6.73 (m, |
| | | | | 1H), 2.29 (s, 3H) |
| 5 | C₁₈H₁₆FN₅O | 337 | 338 | δ 8.76 (s, 2H), 7.93 (dd, J = 7.7, 1.6 Hz, |
| | | | | 1H), 7.72-7.52 (m, 4H), 7.02 (dd, J = |
| | | | | 11.1, 8.4 Hz, 1H), 6.86 (dd, J = 6.8, 4.3 |
| | | | | Hz, 1H), 2.34 (s, 3H) |
| 6 | C₂₁H₁₉N₅O | 357 | | |
| 7 | C₂₂H₁₈FN₃O₂ | 375 | | |
| 8 | C₁₉H₁₆FN₃O | 321 | | |
| 9 | C₁₉H₁₆N₆O | 344 | 345 | δ 11.99 (s, 1H), 10.87 (s, 1H), 9.95 (s, 1H), |
| | | | | 8.63 (d, J = 7.0 Hz, 2H), 7.96 (d, J = |
| | | | | 6.7 Hz, 2H), 7.67 (d, J = 8.4 Hz, 2H), |
| | | | | 7.48 (d, J = 8.4 Hz, 2H), 7.36-7.27 (m, |
| | | | | 2H), 6.84 (dd, J = 5.5, 1.0 Hz, 1H). |

The activity of the compounds in Examples 1-9 as miRNA inhibitors is illustrated in the following assays. The other compounds listed above, which have not yet been made and/or tested, are predicted to have activity in these assays as well.

### Biological Activity Assay

### Linifanib

Linifanib (1-(4-(3-amino-1H-indazol-4-yl)phenyl)-3-(2-fluoro-5-methylphenyl)urea, ABT-869, Example 10, below) has been previously identified as a potential inhibitor of microRNA (Monroig-Bosque, Paloma del C. et al. “OncomiR-10b hijacks the small molecule inhibitor linifanib in human cancers.” Scientific Reports 8, no. 1 (2018): 1-13), and was employed in certain assays as a positive control.

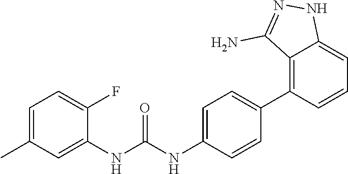 [SMILES: Cc1ccc(F)c(NC(=O)Nc2ccc(-c3cccc4[nH]nc(N)c34)cc2)c1]

### Cell Culture

Cell lines are obtained from the American Type Culture Collection and grown as suggested by the supplier. Experiments are performed using MCF7, MM231, MDA-MB-468, and HepG2 cell lines cultured in DMEM supplemented with 10% Fetal Bovine Serum and maintained at 37° C. in a humidified atmosphere of 5% CO₂. miR-10b-overexpressing MCF7 clones are generated by miR-10b-pcDNA3 plasmid, and the stable clones are selected in media containing G418. MDA-MB-231-FG12-Luc (MM231-FG12-Luc) cells for in vivo imaging is generated by lentiviral infection with FG-12 vector to express both green fluorescent protein and luciferase as previously described (Fuentes-Mattei, E. et al. “Effects of obesity on transcriptomic changes and cancer hallmarks in estrogen receptor-positive breast cancer.” J. National Cancer Institute 106(7) (2014).). MM231-FG12-Luc cells are selected by sorting for green fluorescent protein expression using FACS Aria II (BD Biosciences).

### Cell Line Selection and Maintenance

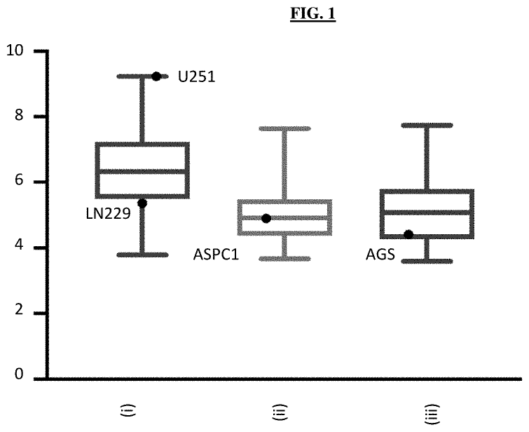

Using the Cancer Cell Line Encyclopedia (CCLE), in-house cancer cell lines were screened to identify cancer and tissue types with varying levels of miR-10b expression level (FIG. 1). Initial screening experiments were conducted using the MCF7 and MDA-MB-231 cancer cell lines, but were shifted to subsequent cell lines for the second phase of this experiment. All experiments were performed using the human-derived cell lines U251 and LN229 (brain cancer), AGS (gastric cancer), and AsPC1 (pancreatic cancer). Two cell lines were chosen for brain cancer analysis to account for PTEN mutation status; PTEN is a tumor suppressor with the most frequent mutation (35-40%) in advanced brain cancer. Cell lines were obtained from the American Type Culture Collection and maintained per instructions from the supplier. U251 was cultured in DMEM supplemented with 10% fetal bovine serum (FBS) and LN229 was cultured in DMEM with 5% FBS. AGS was cultured in F12K medium supplemented with 10% FBS. AsPC1 was cultured in RPMI, with 10% FBS added. All cells were kept at 37° C. in a humidified atmosphere of 5% CO₂, and underwent regular mycoplasma testing. All experiments were conducted when cells were 70-80% confluent.

### Reporter Plasmid Construction

To identify miR-10b inhibitors, a luciferase reporter system was designed with complementary oligonucleotides for miR-10b.This luciferase reporter would repress Renilla luciferase signal in the presence of a functional mature miR-10b. However, in the presence of a small molecule that can inhibit mature miR-10b expression or affect its downstream function, the luciferase expression is restored again.

The PₛᵢCHECK™-2 luciferase reporter vector was acquired from Promega™, Complementary oligonucleotides for hsa-miR-10b were designed based on the mature miRNA sequence and obtained from Thermo Fisher Scientific. In the design of the microRNA target sequence, several base pairs were added to have two different restriction sites recognizable by the enzyme SgfI at one side, and PmeI on the other (present in the vector as well). In addition, the insert was designed with a restriction site for SacI enzyme in order to have an additional site to digest the vector when monitoring colonies for the presence/absence of the insert. The designed oligonucleotides were annealed as follows: Complementary strands were mixed at a 1:1 molar ratio in a micro centrifuge tube. The mixture was diluted to a final concentration of 1 pmol/μl with a buffer solution of 10 mM Tris, 1 mM EDTA, and 50 mM NaCl (pH 8.0). Mixes were incubated at 95° C. for 5 min, and then the heat was reduced gradually for 70 min, until reaching 4° C. The annealed oligonucleotides were digested along with the PsiCHECK™2 vector with SgfI and PmeI and ligated at the 3′-UTR (downstream) of the Renilla luciferase reporter gene in the PsiCHECK™-2vector. Insert sequences were further confirmed in two ways: enzymatic digestion and sequencing.

Molecular-Luciferase-Based Screening of Small Molecule miRNA Inhibitors

MCF7 cells were seeded 24 h prior to transfection in 96 well plates (2.0×10⁴ cells/well). For testing the sensitivity of the assay, antagomiRs and pre-miRs against miR-10b were used at a final concentration of 100 nM. For testing small molecule miRNA inhibitors, the compounds of interest were added to a final concentration of 10 μM. The assay was done in MCF7 or MDA-MB-231 cells at approximately 60% confluence using Lipofectamine in Opti-Mem media with the final vector concentration of 250 ng/mL. All transfections were performed in triplicate for statistical analysis. The cells were incubated at 37° C. for 6 h, followed by replacement of the transfected media with fresh media. After 48 h incubation, the media was removed, and cells were lysed and assayed with a Dual Luciferase Assay Kit (Promega) using a Vi-Tech luminometer. The ratio of Renilla to firefly luciferase expression was calculated for each of the triplicates.

### Quantitative Real Time PCR Analysis (RT-qPCR)

Potential small molecule miRNA inhibitors were tested in MCF7 cells, a metastatic cell line (MM231) and an HCC cell line (HepG2) through RT-qPCR. Cells were seeded in 12-well plates 24 h before treatments at a confluence of 50-60%. They were treated at a concentration of 10 μM linifanib (positive control) or DMSO (negative control), and RNA was collected after 24 and 48 h. MicroRNA expression was tested using TaqMan microRNA assay (Applied Biosystems). The cDNA was synthesized using TaqMan Reverse Transcription Reagents kit (Applied Biosystems) and employed for quantitative RT-qPCR analysis together with TaqMan probes and SsoFastSupermix (Bio-rad). Primers and probes were purchased for hsa-miR-10b, hsa-pri-miR-10b, U6, and U48 snRNA that was used as an internal control.

To detect the levels of the precursor sequences, cDNA was synthesized using the SuperScript III cDNA kit (Invitrogen). Diluted cDNA was used for RT-qPCR analysis using iQ SYBR Green Supermix (Bio-Rad) with primers designed by in-house. Experiments were performed in triplicate; treated samples were compared to the DMSO-treated controls and normalized to the internal control. Relative expression levels were calculated using the 2-ΔCt method.

### Dose-Dependent Cell-Based Assays

The luciferase-reporter (PsiCHECK™-2) vector construct was used to test the half maximal inhibitory concentration (IC₅₀) at which each small molecule miRNA inhibitor induced a response halfway between the baseline and maximum after a specified exposure time. Cells were plated at a 50-60% confluence 24 h before transfection. Transfection were performed as initially done for the primary screen, and effectiveness of small molecule miRNA inhibitors was tested at two separate time-points: 24 and 48 h. A total of five different concentrations were tested in serial dilutions and the IC₅₀ is determined for each compound using MCF7 cells.

### Cell Viability

IC₅₀ dose-response curves were determined using the cell proliferation assay system. Briefly, 5,000 or 10,000 cells of each cell line were plated in 96-well microculture plates in replicates of either 4 or 6. When cells became adherent, either 5 or 10 sequentially decreasing doses of the small molecule miRNA inhibitor were diluted into new media and added to individual wells. After either 24 or 48 hours, of a tetrazolium salt such as MTS, MTT, or WST-8 was added to each well at the same time of day as treatment and incubated for either 2 or 3 h at 37° C. The OD, measuring at the appropriate wavelength for the dye, was read at on a microplate spectrophotometer.

### Lentivector-based MicroRNA Precursor Constructs

Primers were designed for the precursor sequences of each microRNA (OligoPerfect™ Designer) including BamHl and EcoRl/Notl restriction sites, which were then cloned in the pCDH-CMV-MCS-EF1-copGFP vector. For the lentiviral production, 293FT cells were grown in DMEM supplemented with 10% FBS and passed when they were 80% confluent. Cells were transfected in 10 cm dishes when 60% confluent with 8.0 μg of transfer vector (pMirna), 5.2 μg of the packaging vector (CDH-CMV MCS-EF1-copGFP) and 2.8 μg of the envelope vector (pMD.G). After 48 h, the supernatant containing the virus was collected and filtered (0.45 μm). MCF7 cells were then infected with the viral supernatant containing either the empty vector or vector containing the target sequence for miR-10b along with Polybrene/Sequa-brene™ antibiotic, at a final concentration of 8 μg/mL. The plate was then centrifuged at 1500-1800 g for 90 min and incubated at 37° C. for 30 min, and the culture media replaced with fresh one. Transfection efficiency was monitored through GFP, as well as confirmed by RT-qPCR.

### Proliferation Assay

Cell proliferation is determined using a colorimetric assay. A tetrazolium salt such as MTS, MTT, or WST-8 is reduced by dehydrogenase activities in cells to give a formazan dye, which is soluble in the tissue culture media. The amount of the formazan dye (generated by the activities of dehydrogenases in cells, and directly proportional to the number of living cells) is measured after 3 h of incubation. The OD is read at 450 nm on a microplate spectrophotometer and growth values (%) are calculated as (OD treated cells/OD untreated cells)×100. The experiments are performed in triplicate. Cell viability is also tested by bromodeoxyuridine (BrdU) test according to the instructions. 2000 MM231, MCF7 and HepG2 cells are seeded to 96-well plate. After 24 h, 10 μM DMSO or linifanib is added to each well. The OD is read at 450/550 nm on a microplate spectrophotometer, after one to seven days of the treatment. The experiments are performed in triplicate.

### Microsomal Stability Assay

The liver microsomal incubation mixture was composed of liver microsomes (with a concentration of 0.5 mg microsomal protein/ml), the test compound (1 μM), MgCl₂ (3 mM), EDTA (1 mM) in potassium phosphate buffer (100 mM, pH 7.4). Control substrates used were Midazolam and Ketanserin. The reaction was initiated with the addition of an NADPH regeneration solution (1.3 mM NADPH) and maintained at 37° C. with shaking. At five sequential time points ranging from 0 to 45 min, aliquots of the mixture (50 μL) were removed and quenched with acetonitrile (100 μL) containing an internal standard control (imipramine) Samples were analyzed after vortexing and centrifugation by liquid chromatography-mass spectrometry (LC-MS/MS). The in vitro t_{1/2} was then calculated and clearance followed literature.

Detection of Compound Bnding to miRNA

The human pre-miR-10b RNA hairpin sequence (nucleotides 40-75, per miRbase) was transcribed enzymatically and purified using methods previously described. The NMR screening buffer consisted of 20 mM Bis-Tris-d₁₉ at pH 6.5, 10 mM NaCl, 0.2% Tween-20, and 11.1 μM DSA as chemical shift reference (integrates to 100 μM), prepared in 99.99% D₂O or 95%H₂O/5%D₂O. Each ligand was first dissolved in DMSO to a 10 mM stock concentration. Free ligand screening samples were prepared at a final DMSO concentration of 10% at a concentration of 100 μM in NMR screening buffer. For RNA target detected experiments, the lyophilized RNA pellets were dissolved in DMSO-free NMR screening buffer and heated for 4 min at 90° C., then snap cooled for 5 min at −20° C. ¹D-¹H experiments for both ligand and RNA were collected using the Bruker “zgesgp” excitation sculpting pulse sequence to suppress background water signal. Data was collected with 64 scans and 16K data points. Data was processed using 16K zero points, then Fourier transformed followed by manual phase and baseline correction. 2D-¹H TOCSY experiments were collected using the Bruker “mlevesgpph” pulse sequence with excitation sculpting water suppression, data collected with 32 scans, 2K×512 data points, a recycle delay of 1.2 s, and spin-lock mixing time of 80 ms. A reference, ligand free RNA ([pre-miR-10b]=50 μM) TOCSY spectrum was collected prior to adding 4× of 4 (4=200 μM). All data was processed using NMRPipe and visualized using SPARKY. Peaks assignments and secondary structure mapping were completed using methods described previously.

### Clonogenic Assay

In vitro clonogenic assays were conducted with adherent lines seeded in 12-well plates, treated for two weeks, and stained with crystal violet.

### Flow Cytometry

In 6-well plates, 1×10⁵ cells/mL were added into each well. After 24 h, sequential concentrations of DMSO and 4 based were administered and the plates were incubated for 48 h. The supernatant and adherent cells were collected and centrifugated. Each dose was done in triplicate, with each sample composing two wells. For apoptosis analysis, the supernatant was discarded after centrifugation and the pellet washed with PBS twice. Then, BD Annexin V-FITC/PI used to stain the cells for analysis via flow cytometry on the Gallios machine. For cell cycle analysis, after washing with PBS, cells were fixed with ethanol and incubated. They, were treated with RNase enzyme and analyzed.

### Cell Sorting

For co-culturing with cerebral organoids, the brain cancer cell lines U251 and LN229 were transduced with an RFP lentiviral vector expressing mCherry and luciferase, and sorted twice via an Aria Influx Cell Sorter.

### Colony Formation Assay

500 MM231, MCF7, and HepG2 cells are added to 6-well plate. 24 h later, 10 μM DMSO or linifanib is added to each well. Two weeks later, colonies are fixed with 4% paraformaldehyde, stained with crystal violet, and counted using a stereomicroscope.

### In Vitro Migration and Invasion Assay

In the cell motility assay, 100 μL of serum-free media containing 50,000 cells (MM231) are seeded onto the insert with 8.0 μm porous membrane well either coated with gelatin (for migration assay) or Matrigel matrix containing collagen and laminin (for invasion assay), and 500 μL of media with serum is added to the bottom well. Cells are left to migrate or invade for 24 h. The cells that migrated or invaded to the bottom of the well are fixed, stained and counted. For each well, five different fields are counted, and the average number of cells is determined (AutoCAD). The experiments are performed in triplicate. Results from both assays are normalized to proliferation.

### Western Blot

Proteins are collected from cultured cells and lysed with 10× Lysis Buffer (Cell Signaling) freshly supplemented with a complete protease inhibitor cocktail and phosphatase inhibitors (Sigma). Bradford assay is used to measure protein concentration. Proteins are separated by polyacrylamide gel (Bio-rad) electrophoresis and were transferred to a 0.2 μm nitrocellulose membrane (Bio-rad). Quantification of protein expression was conducted using the image analysis software ImageJ.

### Cerebral Organoids

Human induced pluripotent stem cell (iPSC) derived cerebral organoids were obtained, courtesy of Dr. Sanjay Singh and Dr. Frederick Lang in the Department of Neurosurgery at MD Anderson Cancer Center. Organoids were maintained in a specialized neural based media on a shaker inside a 37° C. incubator. After maturation, organoids were moved to 24-well ultra-low attachment plates, and individually co-cultured with 5×10⁵−1×10⁶ cells from brain cancer cell lines for approximately 48-72 h, until RFP signal was clear on the surface of the organoid, indicating cell attachment. Organoids were then moved to 6 well plates and grouped into triplicates within the well. The treatment regimen of control, and 4 was started, at an analogous schedule to in vitro experiments. For fixation and embedding for in situ hybridization, the organoids were processed using 4% PFA and placed in OCT in −80° C. for sectioning.

### Reverse Phase Protein Array (RPPA)

Proteomics analysis was conducted at the Reverse Phase Protein Array Core facility at MD Anderson Cancer Center. Briefly, controls and treated samples were analyzed in triplicate for 486 unique antibodies. The outputs were then normalized for protein loading and transformed to the linear value, and the normalized data was logy transformed (log₂(x+1)) and sorted by FDR-adjusted p-values with a significance level of p<0.05. Differentially expressed proteins between groups were identified using the moderated t-test from LIMMA package. The results of the differential expression analysis (fold changes and p-values) were displayed in a volcano plot, highlighting proteins of interest. Analyses and graphical representations were carried out in R. When preparing the data for analysis, the loge fold change was calculated and adjusted p-value was used for analysis. Gene symbols were converted using the DAVID bioinformatics resource and the clusterProfiler data visualization and analysis package was used for pathway enrichment analysis. For pathway analysis, the Hallmark gene set and Gene Ontology was used for gene set and pathway enrichment.

### In Vivo Experiments

In vivo orthotopic grafting experiments are performed by injecting MM231-FG12-Luc cells in 30 μL of saline solution containing 50% (v/v) of Reduce Growth Hormone Matrigel (BD Bioscience) into the left fourth mammary fat pad of female Nu/Nu mice. In vivo imaging of tumors is performed using a Xenogen IVIS 100 optical in vivo imaging system. The in vivo experiments are conducted in accordance with American Association for Laboratory Animal Science regulations and the approval of The University of Texas MD Anderson Cancer Center Institutional Animal Care & Use Committee.

### Statistical Analysis

All statistical analyses are performed in R (version 3.0.1). All tests are 2-sided and considered statistically significant at the 0.05 level. The RT-qPCR, luciferase dosage dependent analysis, proliferation, migration and invasion assays are performed in triplicate. The MTT and BrdU experiments are performed in quadruplicate. A (two-sided) t-test is applied to compare the mean between control vs. treated samples and analyses are carried out in GraphPad (Prism 6). Log-rank test is used to find the point (cut-off) with the most significant (lowest p-value) split in high vs low miRNA level groups. The Kaplan-Meier plots are generated for these cut-off (0.18). The numbers of patients at risk in low and high miR-10b groups at different time points are presented at the bottom of the graph. Median survival months in each group are presented in brackets. The statistical significance is defined as a P-value less 0.05. All data are represented as standard deviation (S.D.) of the mean.

Luciferase Assay of miR-10b Inhibition

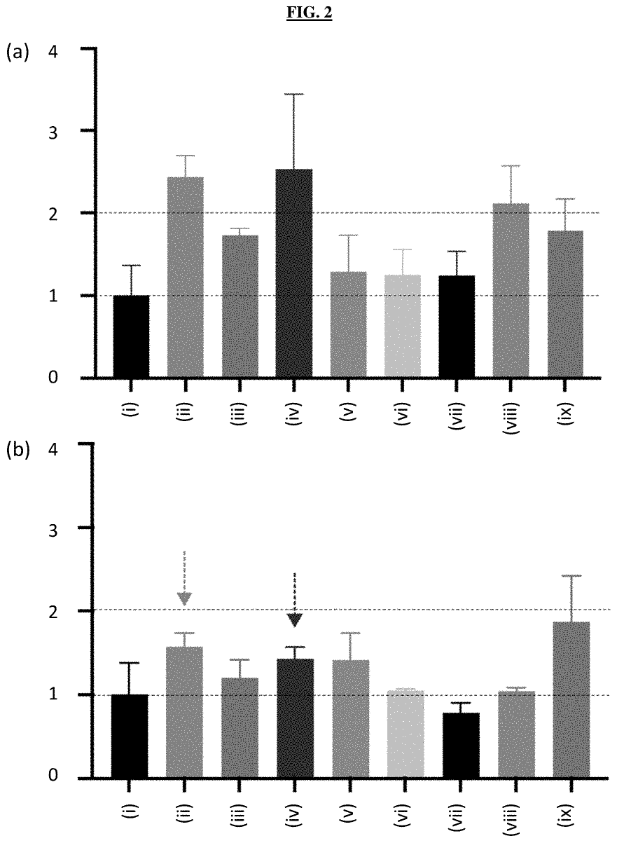

FIG. 2 shows the results of the luciferase assay for (a) MCF7 and (b) MDA-MB-231. (i) negative control (DMSO), (ii) 2, (iii) 6, (iv) 4, (v) 9, (vi) 7, (vii) 8, (viii) 5 (ix) 10. The n-fold increase in luciferase expression (in triplicate, along with mean and population standard deviation), using the Renilla luciferase assay described above, is reported in the following tables.

Tables 3 and 4 report assay results for the MCF7 cell line. Table 5 reports assay results for the MDA-MB-231 cell line. Tables 6 and 7 report assay results for second biological replicants of MCF7 and MDA-MB-231 cell lines, respectively.

TABLE 3
MCF7 Cell Line

| Ex. | | | | | |
|---|---|---|---|---|---|
| No. | #1 | #2 | #3 | Mean | S.D. |
| 1 | | | | | |
| 2 | 2.75042 | 1.778737 | 2.046488 | 2.19 | 0.41 |
| 3 | 0.238113 | 0.246094 | 0.291898 | 0.26 | 0.02 |
| 5 | 1.985544 | 1.488762 | 5.095778 | 2.86 | 1.60 |
| 6 | 2.391339 | 1.413958 | 1.67311 | 1.83 | 0.41 |
| 10 | 2.736856 | 2.519774 | 2.247088 | 2.50 | 0.20 |
| neg | 1.367802 | 0.844075 | 0.788123 | 1 | |

TABLE 4
MCF7 Cell Line

| Ex. | | | | | |
|---|---|---|---|---|---|
| No. | #1 | #2 | #3 | Mean | S.D. |
| 1 | | | | | |
| 4 | 1.952693 | 3.803007 | 2.387321 | 2.71 | 0.79 |
| 7 | 1.337424 | 5.061891 | 0.822444 | 2.41 | 1.89 |
| 8 | 4.601258 | 6.068895 | 1.075826 | 3.92 | 2.10 |
| 9 | 0.939595 | 4.197893 | 1.200103 | 2.11 | 1.48 |
| 10 | 2.558878 | 1.581534 | 1.782222 | 1.97 | 0.42 |
| neg | 0.778778 | 1.076341 | 1.144881 | 1 | |

TABLE 5
MDA-MB-231 Cell Line

| Ex. | | | | | |
|---|---|---|---|---|---|
| No. | #1 | #2 | #3 | Mean | S.D. |
| 1 | | | | | |
| 2 | 1.449421 | 1.899158 | 1.377194 | 1.58 | 0.2 |
| 3 | 0.422586 | 0.642683 | 0.311644 | 0.46 | 0.14 |
| 4 | 1.358247 | 1.698358 | 1.238107 | 1.43 | 0.19 |
| 5 | 1.132105 | 1.012094 | 0.990035 | 1.04 | 0.06 |
| 6 | 0.922199 | 1.620934 | 1.06184 | 1.20 | 0.30 |
| 7 | 1.011752 | 1.085386 | 1.046886 | 1.05 | 0.03 |
| 8 | 0.565715 | 0.991309 | 0.792917 | 0.78 | 0.17 |
| 9 | 1.136761 | 2.053299 | 1.046548 | 1.41 | 0.45 |
| 10 | 1.505547 | 1.144904 | 2.952693 | 1.87 | 0.78 |

TABLE 6
MCF7 Cell Line: Second Biological Replicant

| Ex. | | | | | |
|---|---|---|---|---|---|
| No. | #1 | #2 | #3 | Mean | S.D. |
| 1 | | | | | |
| 2 | 2.507779 | 2.842571 | 1.962952 | 2.44 | 0.36 |
| 3 | 0.257316 | 0.176352 | 0.117372 | 0.18 | 0.06 |
| 4 | 4.278152 | 2.093493 | 1.240165 | 2.54 | 1.28 |
| 5 | 3.032395 | 1.729506 | 1.586993 | 2.12 | 0.65 |
| 6 | 1.715048 | 1.882821 | 1.57756 | 1.73 | 0.12 |
| 7 | 1.86419 | 0.956733 | 0.935014 | 1.25 | 0.43 |
| 8 | 1.816495 | 1.038993 | 0.857434 | 1.24 | 0.42 |
| 9 | 2.165874 | 0.859384 | 0.840083 | 1.29 | 0.62 |
| 10 | 25.62278 | 14.61663 | 13.41221 | 17.88 | 5.49 |

TABLE 7
MDA-MB-231 Cell Line: Second Biological Replicant

| Ex. | | | | | |
|---|---|---|---|---|---|
| No. | #1 | #2 | #3 | Mean | S.D. |
| 1 | | | | | |
| 2 | 2.375009 | 1.876927 | 2.152068 | 2.13 | 0.20 |
| 3 | 0.192782 | 0.148674 | 0.162611 | 0.17 | 0.02 |
| 4 | 1.806415 | 1.796204 | 1.053003 | 1.55 | 0.35 |
| 5 | 0.260177 | 1.574144 | 2.064728 | 1.30 | 0.76 |
| 6 | 1.474376 | 2.524282 | 1.941932 | 1.98 | 0.43 |
| 7 | 1.163972 | 1.309089 | 1.111577 | 1.19 | 0.08 |
| 8 | 1.386609 | 1.003414 | 1.052405 | 1.15 | 0.17 |
| 9 | 1.388941 | 1.614474 | 1.665101 | 1.56 | 0.12 |
| 10 | 2.988955 | 2.241571 | 0.690155 | 1.97 | 0.96 |

Assay of miRNA and Precursors by RT-qPCR

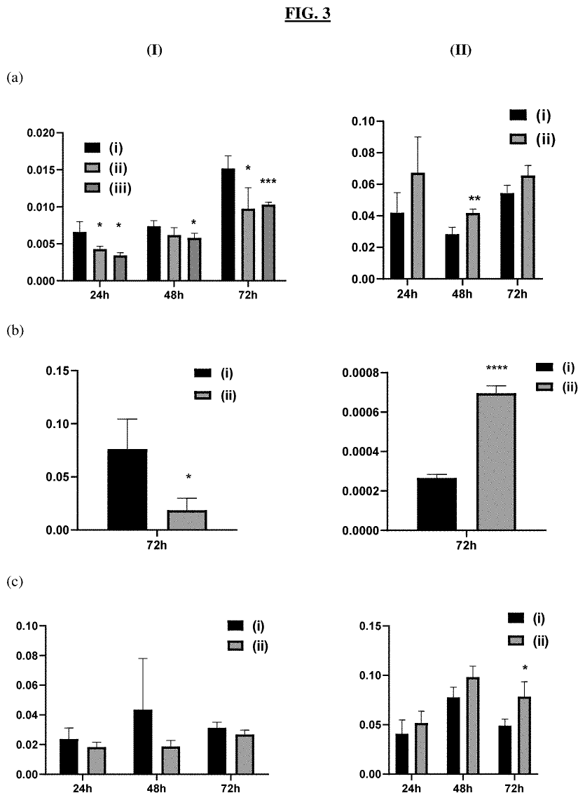

The levels of miR-10b precursors were determined by RT-qPCR. Compounds 2 and 5 reduce the level of mature miR-10b in the AGS gastric cell line by increasing precursor miRNA transcript. FIG. 3(a) shows (I) reduced expression of mature miR-10b, and (II) increased expression of pre-miR-10b, normalized to U6 for (ii) 2 and (iii) 5, as compared to (i) DMSO as a negative control. A second experiment, shown in FIG. 3(b), performed 72 h post-treatment, showed similar results.

Compound 2 reduces the level of mature miR-10b in the AsPC1 pancreatic cell line by increasing precursor miRNA transcript. FIG. 3(c)shows (I) reduced expression of mature miR-10b, and (II) increased expression of pre-miR-10b, normalized to U6 for (ii) 2, as compared to (i) DMSO as a negative control.

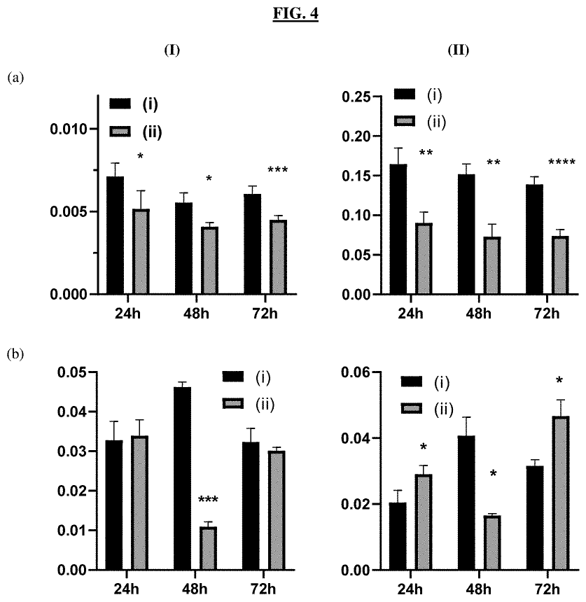

Compound 4 reduces the level of mature miR-10b in certain cell lines by increasing precursor miRNA transcript. FIG. 4 shows the effect of compound 4 on expression of (a) mature miR-10b, and (b) pre-miR-10b, normalized to U6, in the (I) AGS and (II) AsPC1 cell lines, compared to (i) DMSO as a negative control.

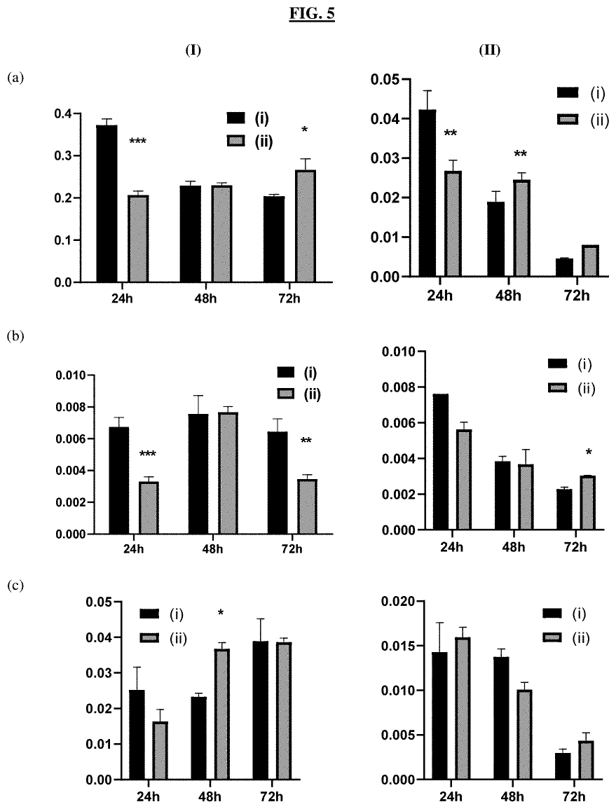

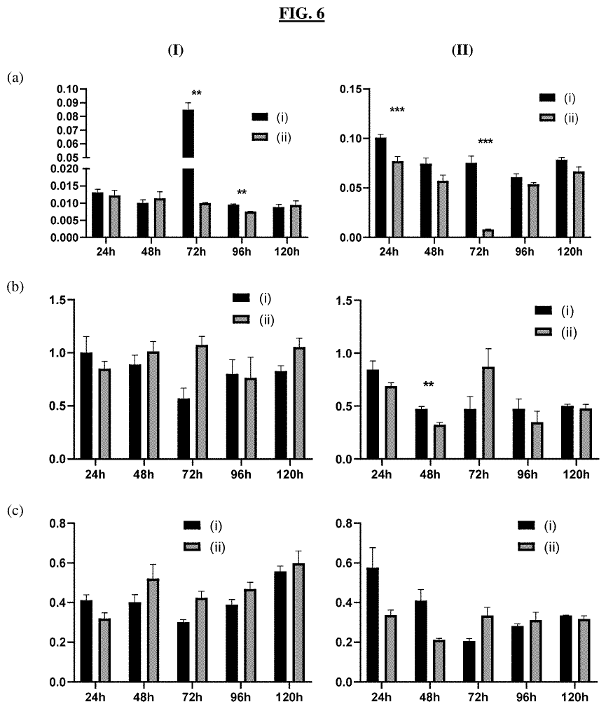

The selectivity of compound 4 for miR-10b becomes apparent upon examination of the effect of compound 4 on other micro-RNA's. FIG. 5 shows the effect of compound 4 on expression of (a) miR-16, (b) miR-28, and (c) miR-182, normalized to U6, in the (I) AGS and (II) AsPC1 cell lines. FIG. 6 shows the effect of compound 4 on expression of (a) miR-10a, (b) miR-21, and (c) miR-155, normalized to U6, in the (I) AGS and (II) AsPC1 cell lines.

### Cell Proliferation Assay

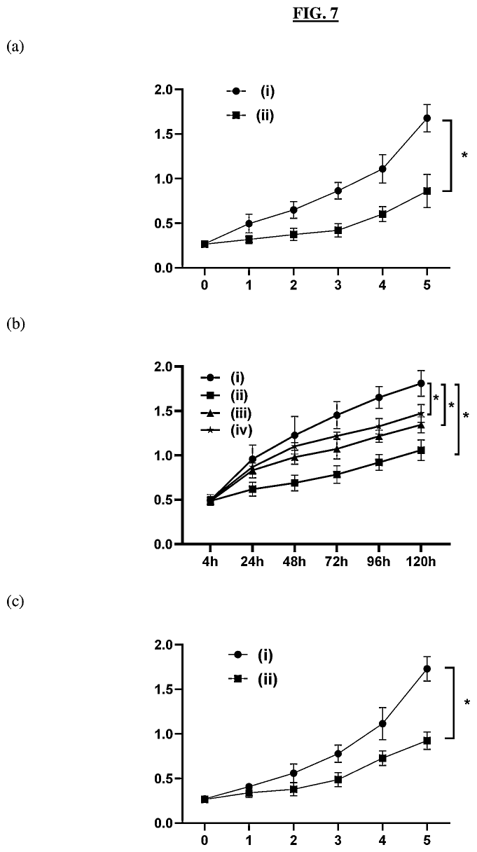

The cell proliferation assay, using either WST-8 or MTS, was used to determine the effect of small molecule miRNA inhibitors on proliferation. Compound 2 reduces proliferation in the AGS and AsPC1 cell lines. FIGS. 7 (a) and (b) show reduced proliferation of the AGS cell line on exposure to (ii) 10 μM, (iii) 5 μM, and (iv) 2 μM of compound 2, compared to (i) DMSO. FIG. 7 (c) shows reduced proliferation of the AsPC1 cell line on exposure to (ii) 10 μM of compound 2, compared to (i) DMSO.

### Microsomal Stability

Compound 4 exhibits favorable microsomal liver stability in human, rat and mouse liver microsomes with a clearance level of 14.9 mL/min/kg, 41 mL/min/kg and 58 mL/min/kg, respectively, and a half-life of 116 min, 60 min and 92 min, respectively (Table 1). These data support the potential use in in vitro and in vivo applications for this compound.

TABLE 8
Microsomal stability

| | Microsomal | | |
|---|---|---|---|
| Compound | Species | Half-life | CLint |
| 4 | Mouse | 92.8 | 58.9 |
| | Rat | 60.4 | 41.1 |
| | Human | 116 | 14.9 |
| 10 | Mouse | 63.1 | 86.5 |
| | Rat | 122 | 20.4 |
| | Human | 197 | 8.8 |

### Cell Viability Assay

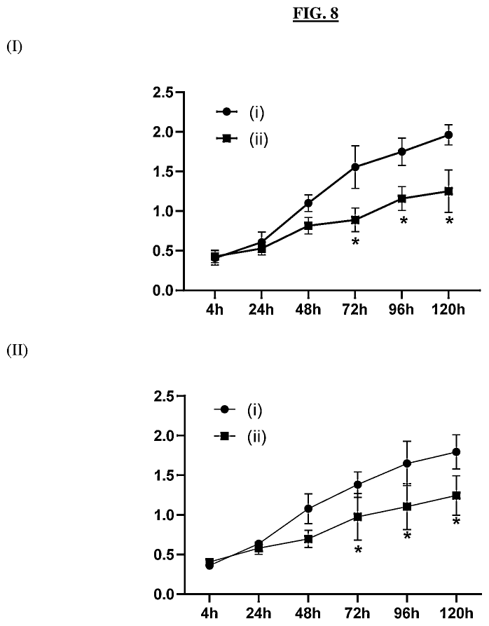

A statistically significant effect is seen in the assay for cell viability. FIG. 8 shows the effect of compound 4 on proliferation ability in the (I) AGS and (II) AsPC1 cell lines. (i) DMSO; (ii) 10 μM 4.

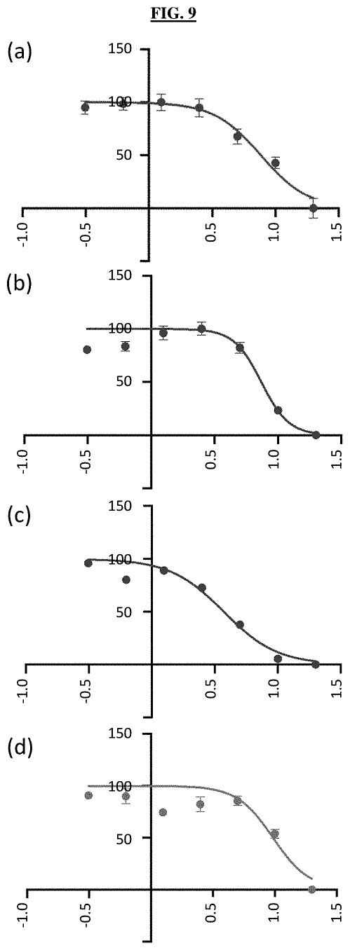

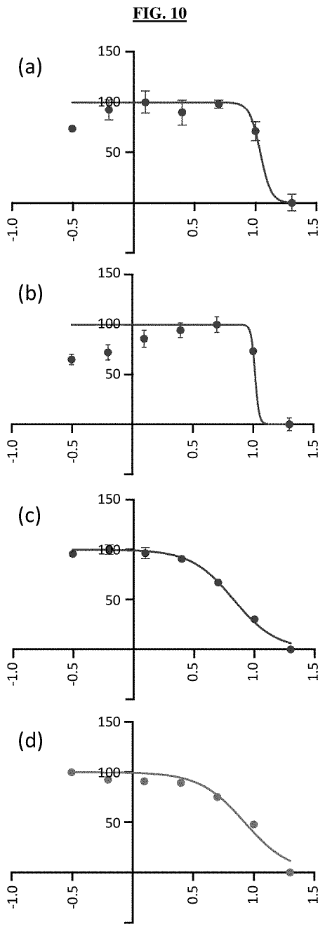

The IC₅₀ values were determined for 4 with each cell line at 24 h and 48 h post-treatment. For the majority of cell lines, the IC₅₀ at 48 h (FIG. 9) was lower than the corresponding IC₅₀ value at 24 h (FIG. 10), and this time interval was thereby chosen for further in vitro experiments.

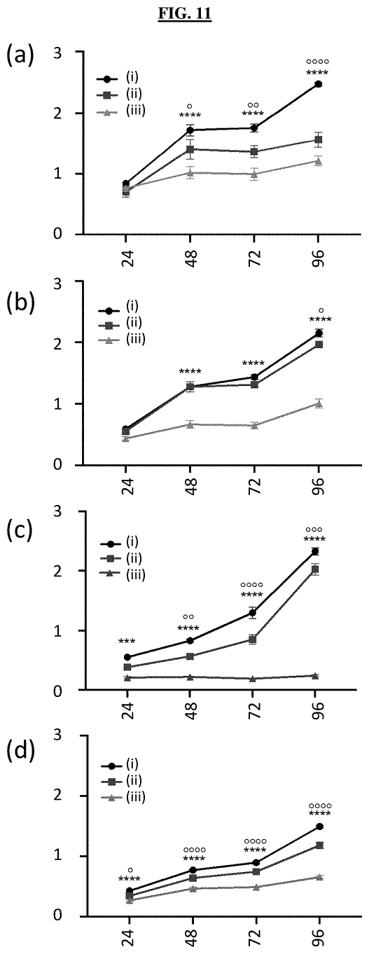

The MTS proliferation assay experiment was conducted in order to evaluate cellular proliferation every 24 h, over a period of 96 h. Each cell line was treated with 5 and 10 μM of 4. As shown in FIG. 11, the two doses of the compound induced a significant difference in proliferation in all cell lines beginning at 48 h of treatment, while several of the cell lines began yielding differences at 24 h.

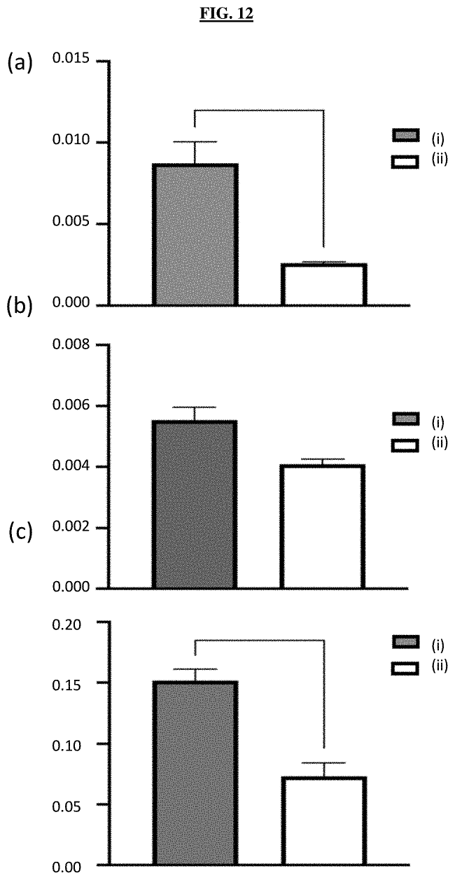

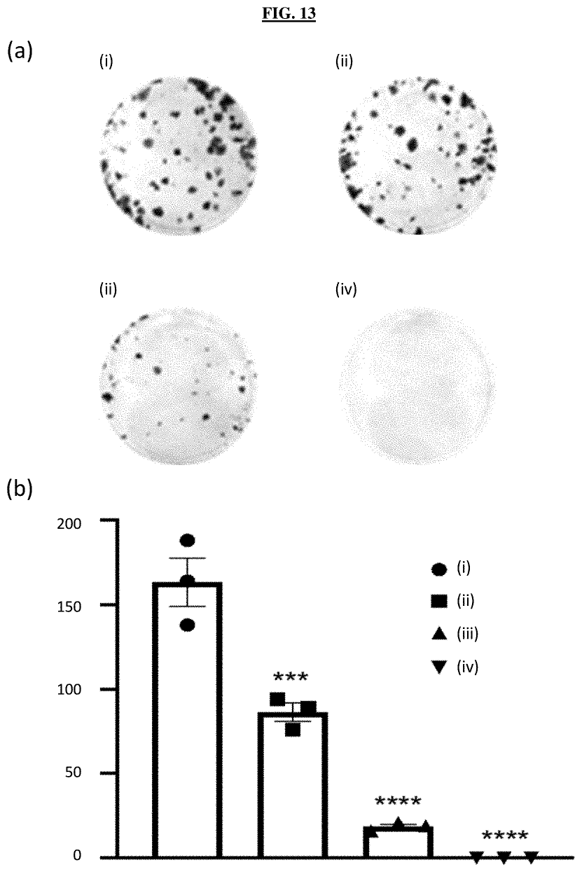

As the U251 brain cancer cell line has the highest expression of miR-10b in our panel of cancer cell lines, several in vitro screenings in this line are reported. The inhibition of miR-10b expression by 4 in the U251 brain cancer cell line, along with the AGS gastric cancer cell line and the AsPC1 pancreatic cancer cell line was examined (FIG. 12). A clonogenic assay was conducted to investigate the formation of colonies after treatment with 4; the compound suppressed the formation of colonies in the cell lines on a dose-dependent basis, yielding a clear decrease in colony formation and eventual elimination of cells beginning at 1 μM of compound up to 5 μM (FIG. 13). These results prove that 4 is effective against malignant cells from multiple histotypes at concentrations that are useful for therapeutic purposes.

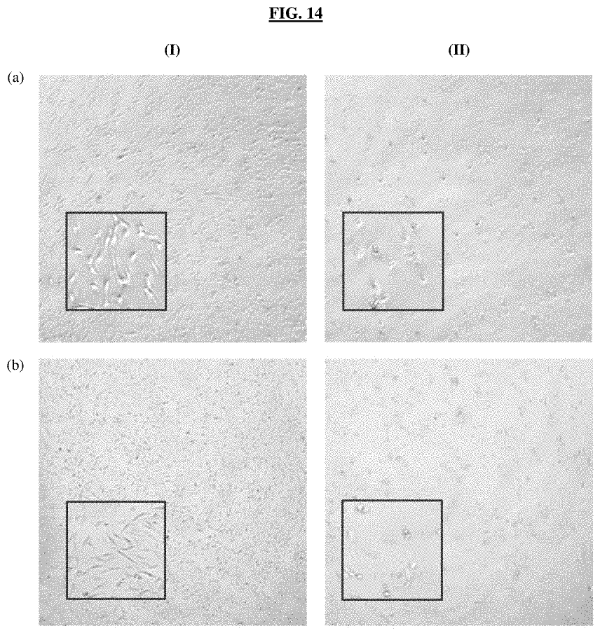

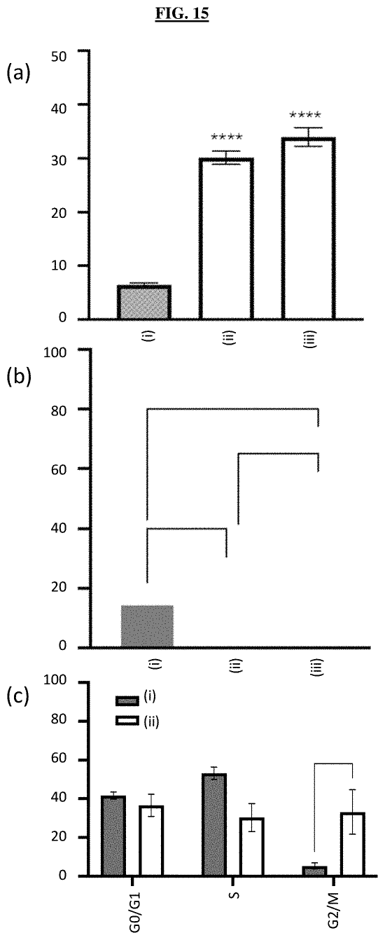

FIG. 14 shows the U251 brain cancer cell line, which has the highest expression of miR-10b in the cell line panel, indicating a significant change in the morphology of the cells over the course of 48 h. The degree of apoptosis was assessed via flow cytometry, with the Annexin V/PI FITC model. As shown in FIG. 15(a), at 5 μM, there was an approximately 4.5-fold increase in total apoptotic cells, compared to control. When evaluated at a dose of 10 μM, the levels were approximately the same, indicating that the rate of apoptosis plateaus after a certain threshold is met. The experiment conducted in the AGS gastric cell line is also shown for comparison in FIG. 15(b), where there is more of a gradual increase up to 10 μM. Cell cycle analysis via flow cytometry is shown in FIG. 15(c). In the U251 brain cancer cell line, it was found that there was a clear increase shown at the G2/M phase. This has been found to be an indicator of cell cycle arrest and potential induction of apoptosis at that stage, which is consistent with our other results.

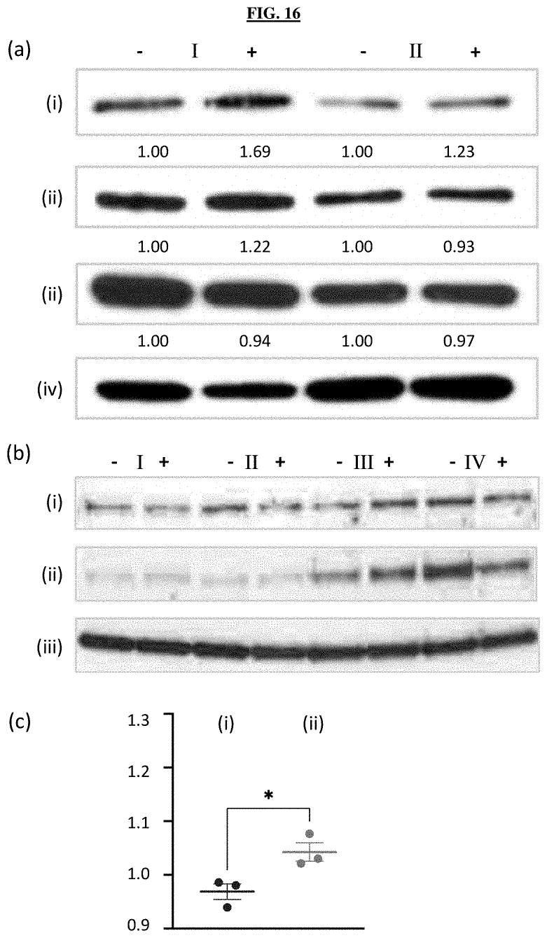

Using the dataset of experimentally validated targets derived from TargetScan and cross-referenced with Oncomine, a preliminary list of miR-10b targets was analyzed for the three cancer types (brain/gastric/pancreatic). The tumor suppressor gene PTEN was the only gene common across the three cancers and was evaluated as a potential target in this study. As shown via Western blots in FIG. 16(a)(i), after treatment with 4, upregulation of PTEN is shown in both brain cancer cell lines: LN229 (I) and U251 (II). This effect is supported by proteomics data, which shows that PTEN expression is significantly upregulated in the U251 brain cancer cell line (FIG. 16(c)). The main targets of the parent compound linifanib, VEGF and PDGF, were assessed. As shown in the Western blots presented in FIGS. 16(a) ii) and (iii), respectively, there is low to very slight variations in the expression of these two compounds in the two brain cancer cell lines. Therefore, 4 has a functional effect through the downregulation of miR-10b and its downstream target modulation, with low variations in the kinase targets of the parent compound, linifanib.

The impact of 4 on the miRNA biogenesis and processing elements Dicer and Drosha was investigated. As shown in the Western blots presented in FIGS. 16(b)(i) and (ii), respectively, little to no variation is shown in the expression of these proteins after treatment with the compound across the four lines and three cancer cell types, indicating there is impact at the precursor level.

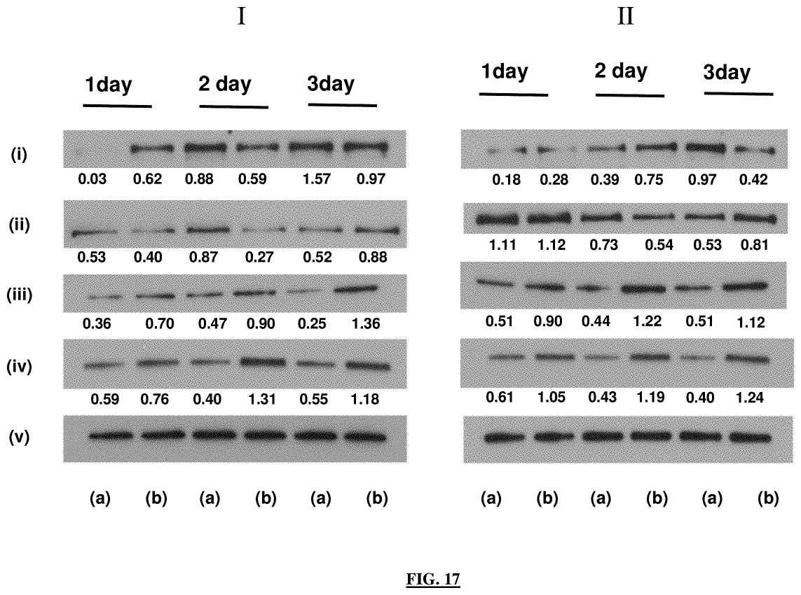

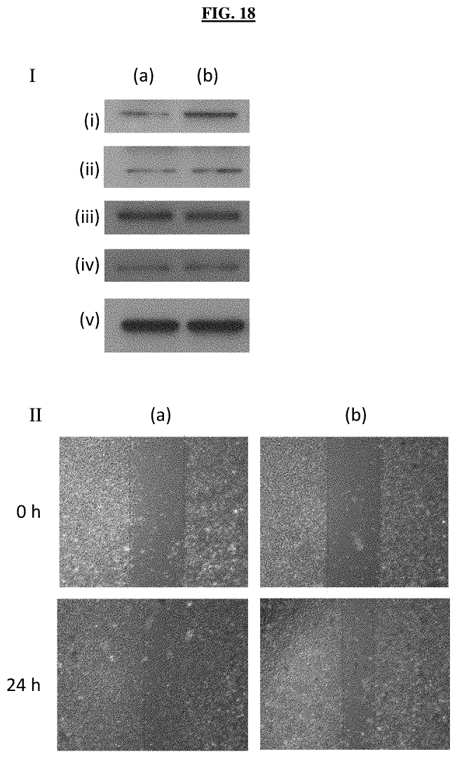

Western blot studies demonstrate that the miR-10b target, HOXD10, is upregulated upon treatment with either of compound 2 or compound 4. FIG. 17 shows Western blots for (I) AGS and (II) AsPC1 cell lines. (a) DMSO; (b) compound 4. (i) Drosha; (ii) Dicer; (iii) PTEN; (iv) HOXD10; (v) β-actin. FIG. 18 (I) shows Western blots for the AGS cell line. (a) DMSO; (b) compound 2. (i) PTEN (ii) HOXD10 (iii) Drosha (iv) Dicer (v) β-actin.

### Migration Assay

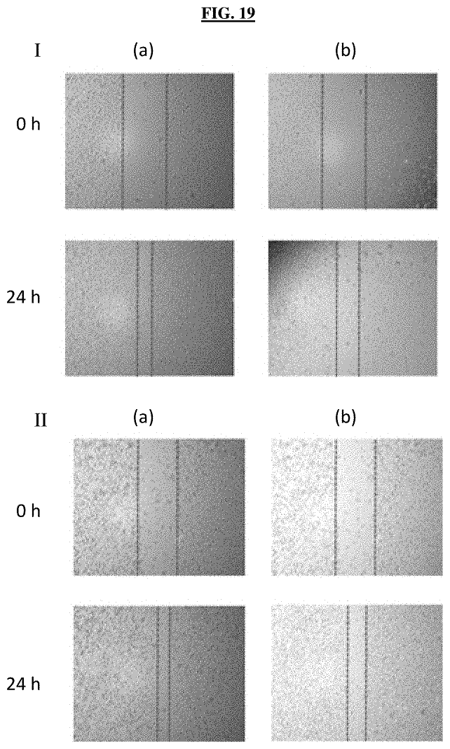

The migration assay shows decreased migration upon treatment with either of compound 2 or compound 4. FIG. 18 (II) shows the effect of compound 2 on migration for the AGS cell line. (a) DMSO; (b) compound 2. FIG. 19 shows the effect of compound 4 on migration for (I) AGS and (II) AsPC1 cell lines. (a) DMSO (b) compound 4.

Compound Binding to miRNA

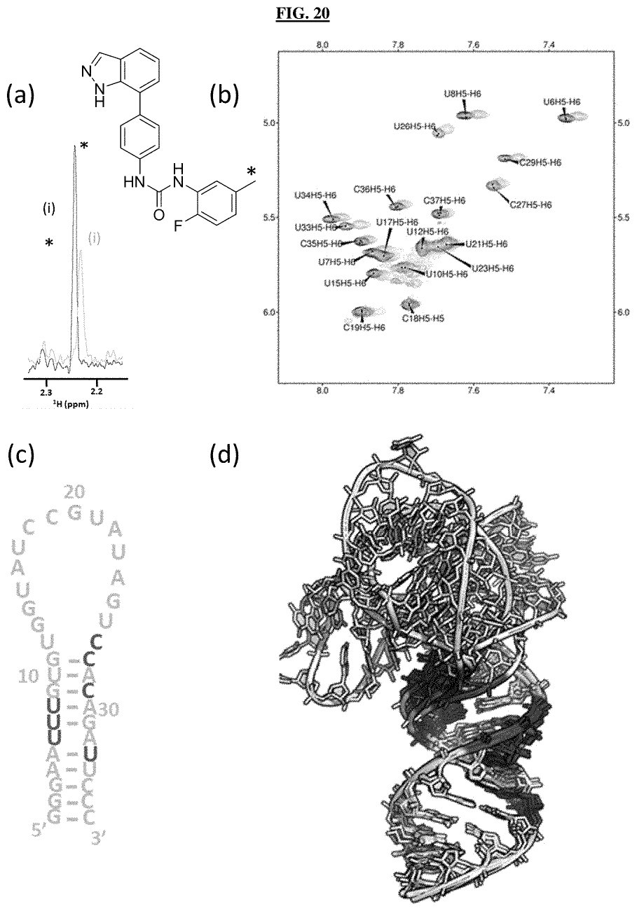

Ligand and target detected NMR experiments were used to assess whether 4 directly binds the apical loop structure common to both the primary-and precursor miR-10b transcripts (FIG. 20). To detect direct binding, first, the ligand detected ¹D-¹H line broadening approach to confirm a direct interaction with the RNA was used (FIG. 20(a)). Non-binding compounds show no change in chemical shift or peak height; therefore, this approach can be used in a binary manner to confirm binding. For 4, we found a large decrease in peak signal (FIG. 20(a)) between the free (black) and bound (grey) ligand samples, particularly the methyl protons (starred), which is indicative of direct binding.

To confirm the direct binding using ligand detect NMR methods, we also ran target detect NMR methods, which monitor changes of the RNA upon titration of the ligand using the 2D ¹H-¹H TOCSY NMR experiment (FIG. 20(b)). In this experiment, 50 μM of pre-miR-10b sequence was titrated with 100 μM of 4. After resonance assignment of the pre-miR-10b apical loop, we were able to identify an approximate binding site for 4. The pyrimidines that showed the largest chemical shift in the ²D-¹H-1H TOCSY experiment were (U6, U7, U8, U26, C27, C29 and U33). From these NMR results, the binding site location of 4 onto the pre-miR-10b structure can be approximated (FIG. 20(c)). Here, the chemical shift changes on to the NMR derived secondary structure and 3D model of pre-miR-10b can be mapped. The 3-dimensional model of pre-miR-10b was created using the NMR-derived secondary structure and proton chemical shifts as structure restraints for the FARFAR2 method (FIG. 20(d)). These data strongly support the hypothesis that 4 directly binds to the hairpin structure of precursor miR-10b.

Of the three cancer types (brain/gastric/pancreatic), brain cancer was chosen to be modeled in this context, since in The Cancer Genome Atlas (TCGA) it was shown that, of these three types, brain cancer had the greatest upregulation of miR-10b in cancer (data not shown). The cell lines U251 and LN229 were co-cultured in a model of cerebral organoids, derived from human induced pluripotent stem cells (iPSCs).

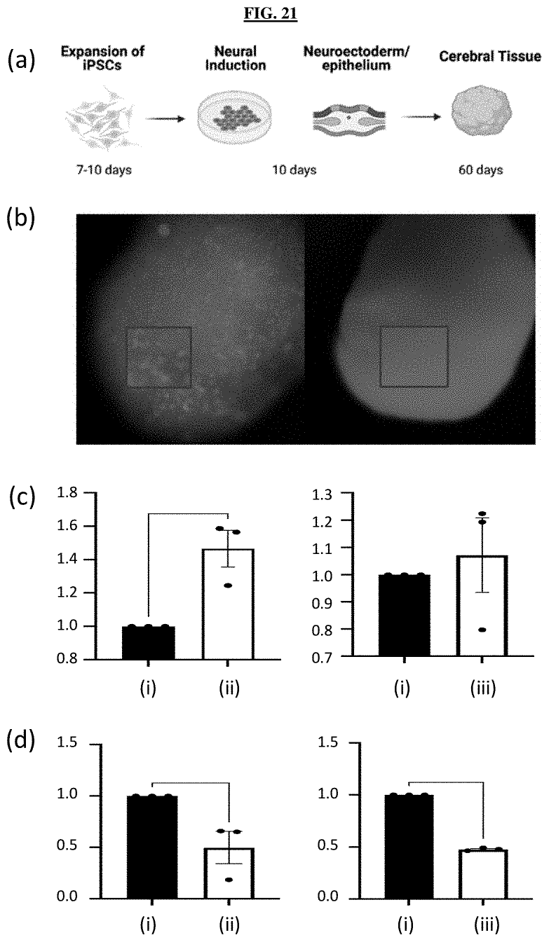

Cerebral organoids are an innovative method to study expression of oncogenic elements of interest, along with novel compounds (FIG. 21(a)). Organoids were analyzed via qRT-PCR, to obtain a baseline level of miR-10b expression. They were then co-cultured with either brain cancer cell line LN229 or U251, and expression of this miRNA was measured via qRT-PCR. Shown are the co-cultured organoids shown under the fluorescent microscope at approximately 48 h and 2 weeks post co-culturing. All of the tumorigenic cells were tagged with red fluorescent protein (RFP) and are easily seen at 48 h. Over the course of two weeks, as they migrated throughout the tissue and proliferated, what can be seen is enhanced bright red areas throughout an organoid, but not specific foci of cells as before. This indicates that the cells had migrated inwards and had begun proliferating, showing that the cell lines were successfully co-cultured with the organoids (FIG. 21(b)). This effect was confirmed, showing a significant increase in miR-10b expression in the co-cultured organoids with LN229 and a trending increase with U251 (FIG. 21(c)).

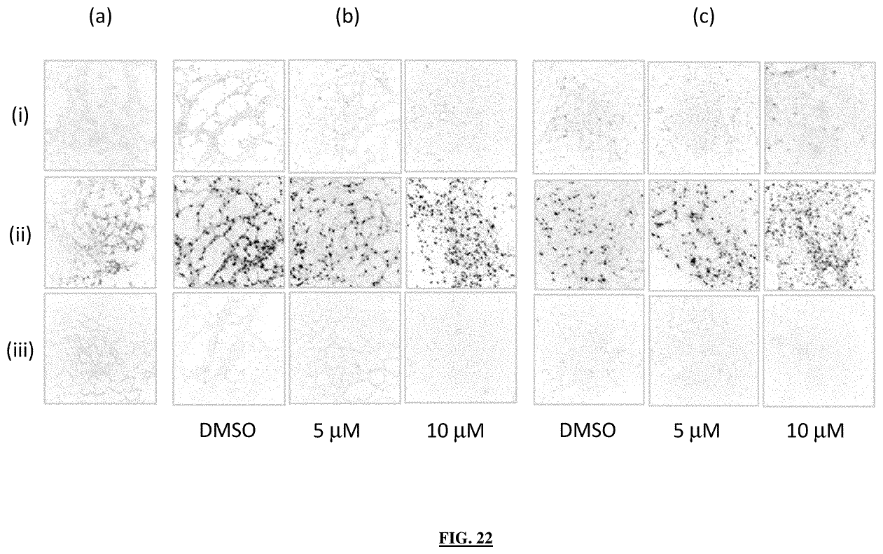

To confirm that 4 yields an impact in this more advanced in vitro model, the co-cultured organoids were treated with varying doses of the compound, and miR-10b levels were measured using qRT-PCR. Significant downregulation of miR-10b was detected with 10 μM of treatment with the compound after 48 h in both of brain cancer cell lines via qRT-PCR (FIG. 21(d)). In situ hybridization was conducted to qualitatively confirm the effect of this treatment on miR-10b expression (FIG. 22). This confirms that that the compound has the potential to work in more models than just biochemical and in vitro, and can potentially be used downstream in higher order models. Therefore, treatment with 4 eliminates malignant cells harboring high levels of miR-10b in cerebral organoids.

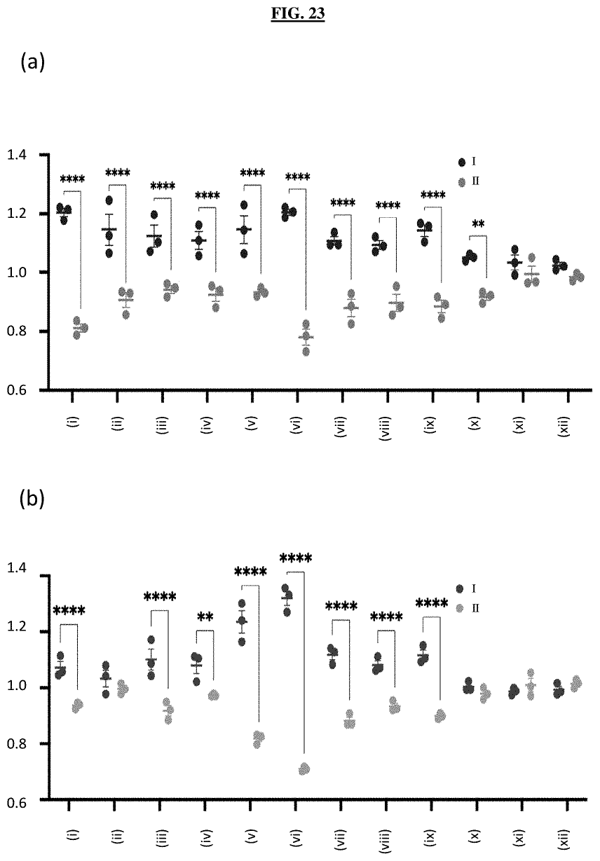

After RPPA analysis, it was found that the majority of proteins on the PI3K/AKT pathway were dysregulated in the tested cell lines; most notably, all of the relevant antibodies targeting proteins on this pathway were downregulated in the U251 brain cancer cell line, with ten of the twelve proteins showing significant downregulation at p<0.01 (FIG. 23(a)).

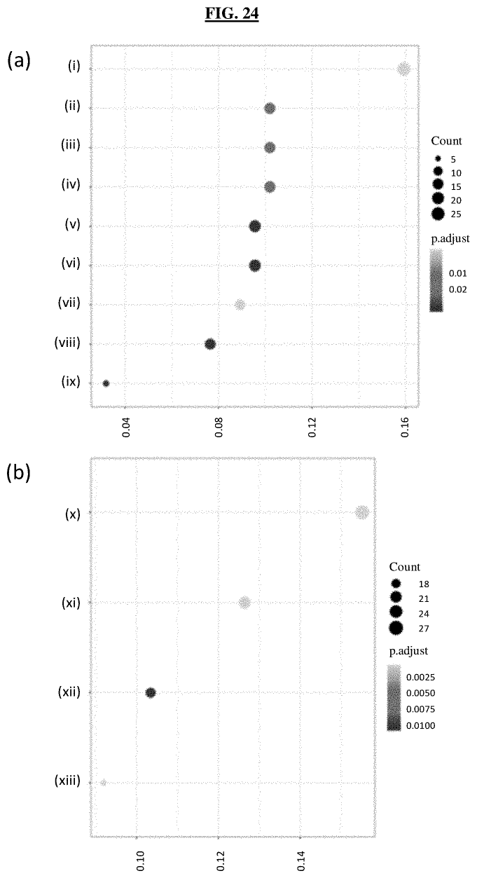

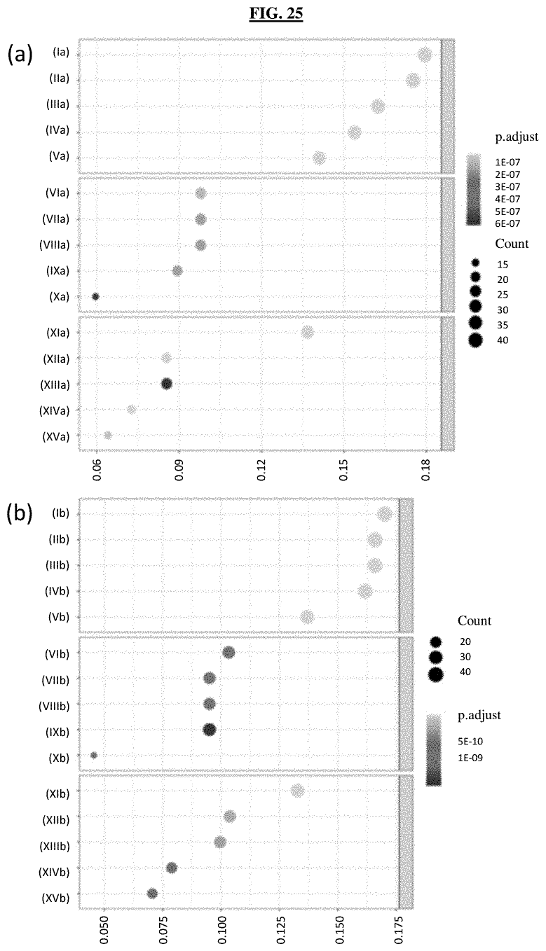

As the dysregulation of the PI3K/AKT pathway has long been established to be oncogenic, these findings indicate a potential mechanistic link between the inhibition of miR-10b and this pathway. This analysis was first conducted on the significantly dysregulated genes in the U251 brain cancer cell line, the line in the panel with the greatest expression of miR-10b, and using the Hallmark gene set, it was found that “Apoptosis” was the pathway most implicated in this change, along with involvement of the “PI3K-AKT-MTOR1 signaling” pathway (FIG. 24). Investigating functional enrichment via Gene Ontology yielded similar results, with “regulation of apoptotic signaling” as one of the primary responses when evaluating the Biological Processes dataset. The result in the U251 brain cancer cell line was compared with the result in the AGS gastric cancer cell line, and it is seen from the Gene Ontology results that apoptosis continues to be a primary hit, with “regulation of apoptotic pathway” and “intrinsic apoptotic signaling pathway” in the top five results when looking at the Biological Processes (FIG. 25). When interrogating the Hallmark gene set for the gastric cell line, “Apoptosis” was also the top hit, with involvement of the “PI3K-AKT-MTOR1 signaling” pathway, indicating that while there are tissue specific effects of this compound on eventual gene expression, ultimately both cancer types are proceeding via the apoptosis pathway.

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this disclosure, and without departing from the spirit and scope thereof, can make various changes and modifications of the disclosure to adapt it to various usages and conditions.

What is claimed is:

1. A compound of structural Formula I: 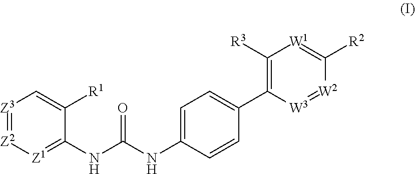
or a salt or tautomer thereof, wherein:
W¹ is chosen from CR⁴ and N;
W² is chosen from CR⁵ and N;
W³ is chosen from CR⁶ and N;
Z¹ is chosen from CR⁷ and N;
Z² is chosen from CR⁸ and N;
Z³ is chosen from CR⁹ and N;
R¹ and R² are independently chosen from H, CN, NH₂, OH, and halo;
R³ is chosen from H, CN, halo, hydroxy, alkyl, and alkoxy;
R⁴ is chosen from H, CN, halo, alkyl, and alkoxy;
or R⁴, if present, and R³, together with the intervening carbons, can form a 5-, 6-, or 7-membered cycloalkyl, heterocycloalkyl, aryl, or heteroaryl ring optionally substituted with one or more R¹⁹;
R⁵, R⁶, R⁷, R⁸, and R⁹ are independently chosen from H, CN, halo, alkyl, and alkoxy; and
each R¹⁹ is independently chosen from CN, halo, OH, NH₂, and oxo.

2. The compound as recited in claim 1, or a salt or tautomer thereof, wherein at least one of W¹, W², W³, Z¹, Z², and Z³ is N.

3. The compound as recited in claim 2, or a salt or tautomer thereof, wherein at least one of W¹, W², and W³ is N.

4. The compound as recited in either one of claims 2 and 3, or a salt or tautomer thereof, wherein at least one of Z¹, Z², and Z³ is N.

5. The compound as recited in claim 1, having structural Formula II: 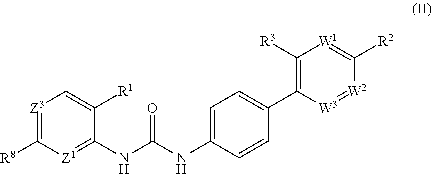
or a salt or tautomer thereof, wherein:
W¹ is chosen from CH and N;
W² is chosen from CR⁵ and N;
W³ is chosen from CR⁶ and N;
Z¹ is chosen from CR⁷ and N;
Z³ is chosen from CR⁹ and N;
R¹ and R² are independently chosen from H, CN, NH₂, OH, and halo;
R³ is chosen from H, CN, halo, hydroxy, alkyl, and alkoxy; and
R⁵, R⁶, R⁷, R⁸, and R⁹ are independently chosen from H, CN, halo, alkyl, and alkoxy.

6. The compound as recited in claim 5, or a salt or tautomer thereof, wherein at least one of W¹, W², and W³ is N.

7. The compound as recited in claim 6, or a salt or tautomer thereof, wherein exactly two of W¹, W², and W³ are N.

8. The compound as recited in claim 7, or a salt or tautomer thereof, wherein:
W¹ and W² are N; and
W³ is CH.

9. The compound as recited in any one of claims 1-8, or a salt or tautomer thereof, wherein Z¹ is CR⁷.

10. The compound as recited in any one of claims 1-9, or a salt or tautomer thereof, wherein Z³ is CR⁹.

11. The compound as recited in any one of claims 1-10, or a salt or tautomer thereof, wherein R⁷ and R⁹ are independently chosen from H, F, and Cl.

12. The compound as recited in any one of claims 1-11, or a salt or tautomer thereof, wherein at most one of R⁷ and R⁹ is not H.

13. The compound as recited in claim 12, or a salt or tautomer thereof, wherein R⁷ and R⁹ are H.

14. The compound as recited in any one of claims 1-13, or a salt or tautomer thereof, wherein R¹ is chosen from H, F, Cl, and Br.

15. The compound as recited in claim 14, or a salt or tautomer thereof, wherein R¹ is chosen from H and F.

16. The compound as recited in any one of claims 1-15, or a salt or tautomer thereof, wherein R² is chosen from H, NH₂, and halo.

17. The compound as recited in claim 16, or a salt or tautomer thereof, wherein R² is chosen from H, NH₂, and F.

18. The compound as recited in claim 17, or a salt or tautomer thereof, wherein R² is H.

19. The compound as recited in any one of claims 1-18, or a salt or tautomer thereof, wherein R³ is chosen from H, halo, and hydroxy.

20. The compound as recited in claim 19, or a salt or tautomer thereof, wherein R³ is chosen from H and F.

21. The compound as recited in claim 20, or a salt or tautomer thereof, wherein R³ is H.

22. The compound as recited in any one of claims 1-21, or a salt or tautomer thereof, wherein R⁸ is chosen from CH₃ and OCH₃.

23. The compound as recited in claim 22, or a salt or tautomer thereof, wherein R⁸ is CH₃.

24. The compound as recited in claim 23, wherein the compound is 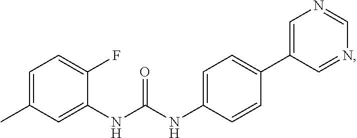 [SMILES: Cc1ccc(F)c(NC(=O)Nc2ccc(-c3cncnc3)cc2)c1]
or a salt or tautomer thereof.

25. The compound as recited in claim 1, having structural Formula IV: 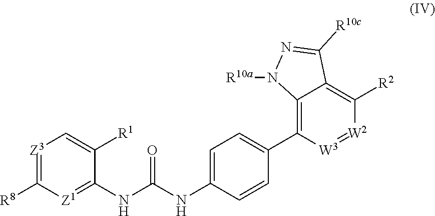
or a salt or tautomer thereof, wherein:
W² is chosen from CR⁵ and N;
W³ is chosen from CR⁶ and N;
Z¹ is chosen from CR⁷ and N;
Z³ is chosen from CR⁹ and N;
R¹ and R² are independently chosen from H, CN, NH₂, OH, and halo;
R⁵, R⁶, R⁷, R⁸, and R⁹ are independently chosen from H, CN, halo, alkyl, and alkoxy; and
R^{10a} and R^{10c} are independently chosen from H, CN, halo, OH, NH₂, and oxo.

26. The compound as recited in claim 25, or a salt or tautomer thereof, wherein at most one of W² and W³ is N.

27. The compound as recited in claim 26, or a salt or tautomer thereof, wherein:
W² is CR⁵, and
W³ is CR⁶.

28. The compound as recited in any one of claims 25-27, or a salt or tautomer thereof, wherein R⁵ and R⁶ are chosen from H and F.

29. The compound as recited in claim 28, or a salt or tautomer thereof, wherein R⁵ and R⁶ are H.

30. The compound as recited in any one of claims 25-29, or a salt or tautomer thereof, wherein Z¹ is CR⁷.

31. The compound as recited in any one of claims 25-30, or a salt or tautomer thereof, wherein Z³ is CR⁹.

32. The compound as recited in any one of claims 25-31, or a salt or tautomer thereof, wherein R⁷ and R⁹ are independently chosen from H and F.

33. The compound as recited in claim 32, or a salt or tautomer thereof, wherein R⁷ and R⁹ are H.

34. The compound as recited in any one of claims 25-33, or a salt or tautomer thereof, wherein R¹ is chosen from H, F, Cl, and Br.

35. The compound as recited in any one of claims 25-34, or a salt or tautomer thereof, wherein R² is chosen from H, NH₂, and F.

36. The compound as recited in claim 35, or a salt or tautomer thereof, wherein R² is H.

37. The compound as recited in any one of claims 25-36, or a salt or tautomer thereof, wherein R⁸ is chosen from CH₃ and OCH₃.

38. The compound as recited in claim 37, or a salt or tautomer thereof, wherein R⁸ is CH₃.

39. The compound as recited in any one of claims 25-18, or a salt or tautomer thereof, wherein R^{10a} is H.

40. The compound as recited in any one of claims 25-39, or a salt or tautomer thereof, wherein R^{10c} is H.

41. The compound as recited in claim 40, wherein the compound is 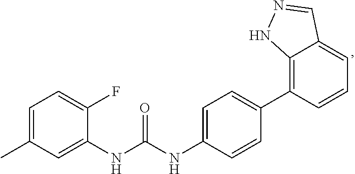 [SMILES: Cc1ccc(F)c(NC(=O)Nc2ccc(-c3cccc4cn[nH]c34)cc2)c1]
or a salt or tautomer thereof.

42. The compound as recited in claim 1, having structural Formula V: 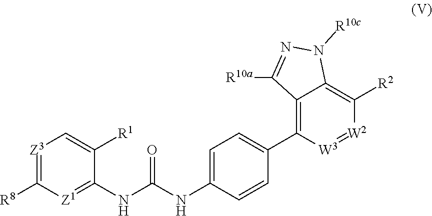
or a salt or tautomer thereof, wherein:
W² is chosen from CR⁵ and N;
W³ is chosen from CR⁶ and N;
Z¹ is chosen from CR⁷ and N;
Z³ is chosen from CR⁹ and N;
R¹ and R² are independently chosen from H, CN, NH₂, OH, and halo;
R⁵, R⁶, R⁷, R⁸, and R⁹ are independently chosen from H, CN, halo, alkyl, and alkoxy; and
R^{10a} and R^{10c} are independently chosen from H, CN, halo, OH, NH₂, and oxo.

43. The compound as recited in claim 42, or a salt or tautomer thereof, wherein at least one of Z¹ and Z³ is N.

44. The compound as recited in either one of claims 42 and 43, or a salt or tautomer thereof, wherein:
W² is CR⁵, and
W³ is CR⁶.

45. The compound as recited in any one of claims 42-44, or a salt or tautomer thereof, wherein R⁵ and R⁶ are H.

46. The compound as recited in any one of claims 42-45, or a salt or tautomer thereof, wherein R⁷ and R⁹ are H.

47. The compound as recited in any one of claims 42-46, or a salt or tautomer thereof, wherein R^{10c} is H.

48. The compound as recited in any one of claims 42-47, or a salt or tautomer thereof, wherein R^{10a} is H.

49. The compound as recited in any one of claims 42-47, or a salt or tautomer thereof, wherein R^{10a} is NH2.

50. The compound as recited in claim 1, having structural Formula VI: 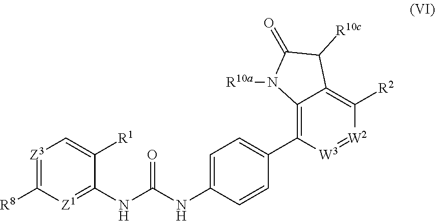
or a salt or tautomer thereof, wherein:
W² is chosen from CR⁵ and N;
W³ is chosen from CR⁶ and N;
Z¹ is chosen from CR⁷ and N;
Z³ is chosen from CR⁹ and N;
R¹ and R² are independently chosen from H, CN, NH₂, OH, and halo;
R⁵, R⁶, R⁷, R⁸, and R⁹ are independently chosen from H, CN, halo, alkyl, and alkoxy; and
R^{10a} and R^{10c} are independently chosen from H, CN, halo, OH, NH2, and oxo.

51. The compound as recited in claim 50, or a salt or tautomer thereof, wherein Z¹ and Z³ are CH.

52. The compound as recited in either one of claims 51 and 52, or a salt or tautomer thereof, wherein W² and W³ are CH.

53. The compound as recited in any one of claims 50-52, or a salt or tautomer thereof, wherein R^{10a} and R^{10c} are H.

54. The compound as recited in claim 1, chosen from 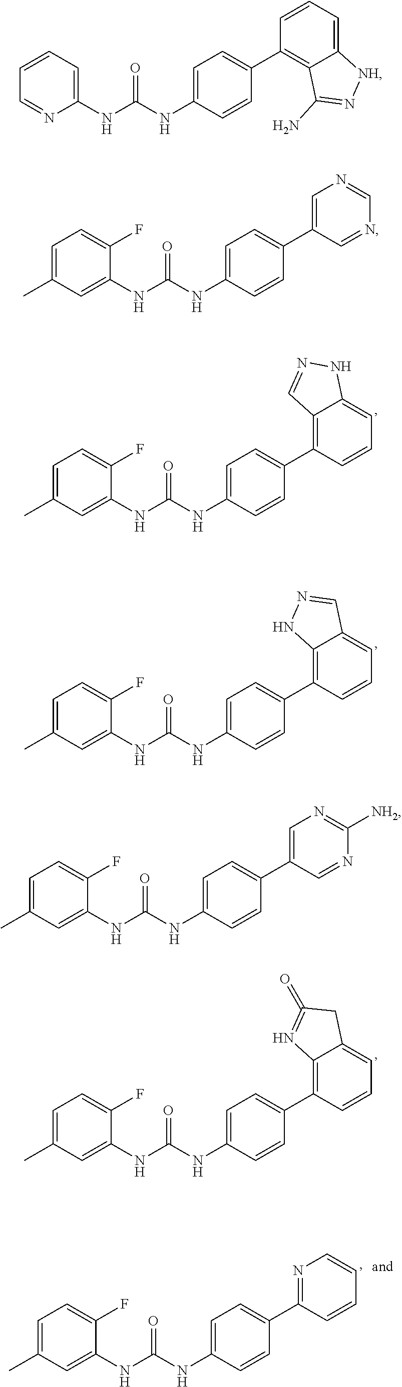 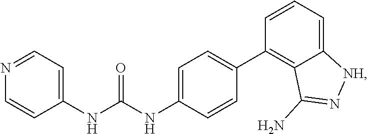 [SMILES: Nc1n[nH]c2cccc(-c3ccc(NC(=O)Nc4ccncc4)cc3)c12]
or a salt or tautomer thereof.

55. A compound as recited in any one of claims 1-54, or a salt or tautomer thereof, for use as a medicament.

56. A compound as recited in any one of claims 1-54, or a salt or tautomer thereof, for use in the treatment of cancer.

57. A compound as recited in any one of claims 1-54, or a salt or tautomer thereof, for use in the manufacture of a medicament for the prevention or treatment of a disease or condition ameliorated by the inhibition of miRNA.

58. A pharmaceutical composition comprising a compound as recited in any one of claims 1-54, or a salt or tautomer thereof, together with a pharmaceutically acceptable carrier.

59. A method of inhibition of miRNA expression comprising contacting miRNA with a compound as recited in any one of claims 1-54, or a salt or tautomer thereof.

60. A method of treatment of a miRNA-mediated disease comprising the administration of a therapeutically effective amount of a compound as recited in any one of claims 1-54, or a salt or tautomer thereof, to a patient in need thereof.

61. The method as recited in claim 60, wherein said disease is cancer.

62. The method as recited in claim 61, wherein said cancer is chosen from acoustic neuroma, acute leukemia, acute lymphocytic leukemia, acute myelocytic leukemia, acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, diffuse large B-cell lymphoma, dysproliferative changes, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, head and neck cancer, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor.

63. The method as recited in in claim 61, further comprising the administration of a non-chemical method of cancer treatment.

64. The method as recited in in claim 63, wherein the non-chemical method of cancer treatment is chosen from surgery, radiation therapy, thermoablation, focused ultrasound therapy, and cryotherapy.

65. The method as recited in in claim 64, wherein the non-chemical method of cancer treatment is radiotherapy.

66. A method of treatment of a miRNA-mediated disease comprising the administration of:
(a) a therapeutically effective amount of a compound as recited in any one of claims 1-54, or a salt or tautomer thereof; and
(b) another therapeutic agent.

67. The method as recited in claim 66, wherein said disease is cancer.
